(19) United States
(12) Patent Application Publication
(10) Pub. No.: US 2025/0185883 A1
(43) Pub. Date: Jun. 12, 2025

(54) MEDICAL SUPPORT DEVICE, ENDOSCOPE APPARATUS, MEDICAL SUPPORT METHOD, AND PROGRAM

(71) Applicant: FUJIFILM CORPORATION, Tokyo (JP)

(72) Inventor: Rito MURASE, Kanagawa (JP)

(21) Appl. No.: 18/956,012

(22) Filed: Nov. 22, 2024

(30) Foreign Application Priority Data
Dec. 6, 2023 (JP) 2023-206458
Oct. 28, 2024 (JP) 2024-189239

(51) Int. Cl. A61B 1/00; A61B 1/31; G06V 10/26; G06V 10/774

(52) U.S. Cl. CPC A61B 1/000096; G06V 10/26; G06V 10/774; A61B 1/31; G06V 2201/03

(57) ABSTRACT

A medical support method includes: causing a trained model to generate certainty information in which a certainty of a lumen being present in each of a plurality of divided regions obtained by dividing a medical image or an image corresponding to the medical image in a circumferential direction is given to the plurality of divided regions; outputting first information indicating that the lumen is present in any of the plurality of divided regions on the basis of the certainty information; and outputting second information indicating that the lumen is present in a central region of the medical image in a case where the certainty information is information in which a value exceeding a threshold value is given as the certainty to two or more divided regions having a positional relationship exceeding 90 degrees in the circumferential direction among the plurality of divided regions.

### CROSS-REFERENCE TO RELATED APPLICATIONS

This application claims priority under 35 USC 119 from Japanese Patent Application No. 2023-206458, filed on Dec. 6, 2023, and Japanese Patent Application No. 2024-189239, filed on Oct. 28, 2024, the disclosures of which are incorporated herein by reference in their entireties.

### BACKGROUND

### 1. Technical Field

The present disclosure relates to a medical support device, an endoscope apparatus, a medical support method, and a program.

### 2. Related Art

JP2003-093328A discloses an endoscope insertion direction detection method comprising a first step of inputting an endoscope image, a second step of detecting a direction in which light and dark change in the endoscope image, and a third step of generating information related to an insertion direction of an endoscope on the basis of a detection result. In addition, JP2003-093328A also discloses an endoscope insertion direction detection method comprising a first step of setting candidate insertion directions which are candidates for an insertion direction of an endoscope, a second step of inputting an endoscope image, a third step of detecting a direction in which light and dark change in the endoscope image, a fourth step of evaluating a similarity between a plurality of candidate insertion directions and the direction in which light and dark change, and a fifth step of determining the insertion direction of the endoscope on the basis of an evaluation result.

WO2020/194472A discloses a movement support system comprising: a multiple-operation information calculation unit that calculates multiple-operation information indicating a plurality of operations, which are different in time and correspond to a multiple-operation target scene which is a scene requiring a plurality of operations different in time, on the basis of a captured image acquired by an imaging unit disposed in an insertion portion; and a presentation information generation unit that generates presentation information for the insertion portion on the basis of the multiple-operation information calculated by the multiple-operation information calculation unit.

### SUMMARY

An embodiment according to the present disclosure provides a medical support device, an endoscope apparatus, a medical support method, and a program that enable a user or the like to ascertain a position of a lumen, which is included in a medical image, in the medical image with high accuracy.

According to a first aspect of the present disclosure, there is provided a medical support device comprising a processor. The processor is configured to: input a medical image generated by imaging an inside of a luminal organ including a lumen to a trained model to generate certainty information in which a certainty of the lumen being present in each of a plurality of divided regions obtained by dividing the medical image or an image corresponding to the medical image in a circumferential direction is given to the plurality of divided regions; and perform an output process capable of distinguishing between a case where the certainty information is not information in which a value exceeding a threshold value is given as the certainty to two or more divided regions having a positional relationship exceeding 90 degrees in the circumferential direction among the plurality of divided regions, and a case where the certainty information is information in which a value exceeding the threshold value is given as the certainty to two or more divided regions having a positional relationship exceeding 90 degrees in the circumferential direction among the plurality of divided regions.

According to a second aspect of the present disclosure, in the medical support device according to the first aspect, the output process may include outputting first information indicating that the lumen is present in any of the plurality of divided regions based on the certainty information in a case where the certainty information is not information in which a value exceeding the threshold value is given as the certainty to two or more divided regions having a positional relationship exceeding 90 degrees in the circumferential direction among the plurality of divided regions, and not outputting the first information in a case where the certainty information is information in which a value exceeding the threshold value is given as the certainty to two or more divided regions having a positional relationship exceeding 90 degrees in the circumferential direction among the plurality of divided regions.

According to a third aspect of the present disclosure, in the medical support device according to the first aspect, the output process may include the output process includes outputting second information indicating that the lumen is present in a central region of the medical image, or not outputting the second information, in a case where the certainty information is information in which a value exceeding the threshold value is given as the certainty to two or more divided regions having a positional relationship exceeding 90 degrees in the circumferential direction among the plurality of divided regions.

According to a fourth aspect of the present disclosure, in the medical support device according to the first aspect, the output process may include not outputting the second information in a case where the certainty information is not information in which a value exceeding the threshold value is given as the certainty to two or more divided regions having a positional relationship exceeding 90 degrees in the circumferential direction among the plurality of divided regions.

According to a fifth aspect of the present disclosure, in the medical support device according to the third aspect, the output process may include outputting the second information in a case where the certainty information is information in which the value is given to two or more divided regions that are disposed at equal intervals around a center of the medical image or the image and have a positional relationship exceeding 90 degrees in the circumferential direction among the plurality of divided regions.

According to a sixth aspect of the present disclosure, in the medical support device according to the third aspect, the output process may include outputting the second information in a case where the certainty information is information in which the value is given to two or more divided regions having a positional relationship of 120 degrees or more in the circumferential direction among the plurality of divided regions.

According to a seventh aspect of the present disclosure, in the medical support device according to the third aspect, the output process may include outputting the second information in a case where the certainty information is information in which the value is given to two or more divided regions disposed at regular intervals around an entire circumference of the medical image or the image among the plurality of divided regions.

According to an eighth aspect of the present disclosure, in the medical support device according to the third aspect, the output process may include output the second information in a case where the certainty information is information in which the value is given to all of the plurality of divided regions.

According to a ninth aspect of the present disclosure, in the medical support device according to any one of the second to eighth aspects, the output process may include outputting the first information in a case where the certainty information is information in which the value is given to a single divided region among the plurality of divided regions and in a case where the certainty information is information in which the value is given to two or more divided regions having a positional relationship of 90 degrees or less in the circumferential direction among the plurality of divided regions.

According to a tenth aspect of the present disclosure, in the medical support device according to any one of the first to ninth aspects, each of the plurality of divided regions may be a region obtained by radially dividing the medical image.

According to an eleventh aspect of the present disclosure, in the medical support device according to the tenth aspect, the plurality of divided regions may be eight divided regions that are present in a radial shape.

According to a twelfth aspect of the present disclosure, in the medical support device according to any one of the first to eleventh aspects, the trained model may be obtained by machine learning using training data including an example image, which indicates a sample of the medical image and is divided into a plurality of regions corresponding to the plurality of divided regions, and correct answer data associated with the example image, the correct answer data in a case where a sample of the lumen is included in a region other than a central region of the example image may be an annotation capable of specifying a position of the region including the sample of the lumen among the plurality of regions, and the correct answer data in a case where the sample of the lumen is included in the central region of the example image may be an annotation capable of specifying positions of all of the plurality of regions.

According to a thirteenth aspect of the present disclosure, in the medical support device according to any one of the first to twelfth aspects, the output process may include: displaying first information indicating that the lumen is present in any of the plurality of divided regions based on the certainty information on a screen to output the first information in a case where the certainty information is not information in which a value exceeding a threshold value is given as the certainty to two or more divided regions having a positional relationship exceeding 90 degrees in the circumferential direction among the plurality of divided regions; and displaying second information indicating that the lumen is present in a central region of the medical image on the screen to output the second information in a case where the certainty information is information in which a value exceeding a threshold value is given as the certainty to two or more divided regions having a positional relationship exceeding 90 degrees in the circumferential direction among the plurality of divided regions.

According to a fourteenth aspect of the present disclosure, in the medical support device according to the thirteenth aspect, the processor may be configured to: display the medical image on the screen; display the first information in the medical image displayed on the screen; and display the second information in the medical image displayed on the screen.

According to a fifteenth aspect of the present disclosure, in the medical support device according to any one of the first to fourteenth aspects, the output process includes outputting first information indicating that the lumen is present in any of the plurality of divided regions based on the certainty information in a case where the certainty information is not information in which a value exceeding the threshold value is given as the certainty to two or more divided regions having a positional relationship exceeding 90 degrees in the circumferential direction among the plurality of divided regions, and outputting second information indicating that the lumen is present in a central region of the medical image in a case where the certainty information is information in which a value exceeding the threshold value is given as the certainty to two or more divided regions having a positional relationship exceeding 90 degrees in the circumferential direction among the plurality of divided regions; and the first information and the second information may be visible information capable of visually specifying a position of the lumen included in the medical image.

According to a sixteenth aspect of the present disclosure, in the medical support device according to any one of the first to fifteenth aspects, the medical image may be an endoscope image generated by imaging the inside of the luminal organ including the lumen with an endoscope.

According to a seventeenth aspect of the present disclosure, there is provided an endoscope apparatus comprising: the medical support device according to any one of the first to sixteenth aspects; and an endoscope. The medical image is generated by imaging the inside of the luminal organ including the lumen with the endoscope.

According to a eighteenth aspect of the present disclosure, there is provided a medical support method comprising: inputting a medical image generated by imaging an inside of a luminal organ including a lumen to a trained model to generate certainty information in which a certainty of the lumen being present in each of a plurality of divided regions obtained by dividing the medical image or an image corresponding to the medical image in a circumferential direction is given to the plurality of divided regions; and performing an output process capable of distinguishing between a case where the certainty information is not information in which a value exceeding a threshold value is given as the certainty to two or more divided regions having a positional relationship exceeding 90 degrees in the circumferential direction among the plurality of divided regions, and a case where the certainty information is information in which a value exceeding the threshold value is given as the certainty to two or more divided regions having a positional relationship exceeding 90 degrees in the circumferential direction among the plurality of divided regions.

According to a nineteenth aspect of the present disclosure, there is provided a program causing a computer to execute a process comprising: inputting a medical image generated by imaging an inside of a luminal organ including a lumen to a trained model to generate certainty information in which a certainty of the lumen being present in each of a plurality of divided regions obtained by dividing the medical image or an image corresponding to the medical image in a circumferential direction is given to the plurality of divided regions; and performing an output process capable of distinguishing between a case where the certainty information is not information in which a value exceeding a threshold value is given as the certainty to two or more divided regions having a positional relationship exceeding 90 degrees in the circumferential direction among the plurality of divided regions, and a case where the certainty information is information in which a value exceeding the threshold value is given as the certainty to two or more divided regions having a positional relationship exceeding 90 degrees in the circumferential direction among the plurality of divided regions.

### BRIEF DESCRIPTION OF THE DRAWINGS

Exemplary embodiments of the technology of the disclosure will be described in detail based on the following figures, wherein:

FIG. 1 is a conceptual diagram illustrating an example of an aspect in which an endoscope apparatus is used by a doctor;

FIG. 2 is a conceptual diagram illustrating an example of an overall configuration of the endoscope apparatus;

FIG. 3 is a block diagram illustrating an example of a hardware configuration of an electrical system of the endoscope apparatus;

FIG. 4 is a block diagram illustrating an example of functions of main units of a processor included in a medical support device and an example of information stored in a storage;

FIG. 5 is a block diagram illustrating an example of a hardware configuration of an electrical system of an information processing apparatus;

FIG. 6 is a conceptual diagram illustrating an example of an aspect in which training data is generated by the information processing apparatus;

FIG. 7 is a conceptual diagram illustrating a comparative example of an example image;

FIG. 8 is a conceptual diagram illustrating a comparative example of training data generated in a case where a lumen is included in a region other than a central region of the example image illustrated in FIG. 7;

FIG. 9 is a conceptual diagram illustrating a comparative example of training data generated in a case where the lumen is included in the central region of the example image illustrated in FIG. 7;

FIG. 10 is a conceptual diagram illustrating an example of processing content in the information processing apparatus in a case where a lumen recognition model is generated by performing machine learning using training data on a model;

FIG. 11 is a conceptual diagram illustrating an example of an example image according to an embodiment;

FIG. 12 is a conceptual diagram illustrating an example of training data generated in a case where the lumen is imaged in a region other than a central region of the example image illustrated in FIG. 11;

FIG. 13 is a conceptual diagram illustrating an example of training data generated in a case where the lumen is included in the central region of the example image illustrated in FIG. 11;

FIG. 14 is a conceptual diagram illustrating an example of processing content of a recognition unit of the medical support device;

FIG. 15 is a conceptual diagram illustrating an example of certainty information obtained in a case where the lumen is included in a region other than a central region of a frame;

FIG. 16 is a conceptual diagram illustrating an example of processing content of a controller and an example of display content of a screen in a case where the lumen is included in a region other than the central region of the frame;

FIG. 17 is a conceptual diagram illustrating a first example of the certainty information obtained in a case where the lumen is included in the central region of the frame;

FIG. 18 is a conceptual diagram illustrating a second example of the certainty information obtained in a case where the lumen is included in the central region of the frame;

FIG. 19 is a conceptual diagram illustrating a third example of the certainty information obtained in a case where the lumen is included in the central region of the frame;

FIG. 20 is a conceptual diagram illustrating an example of the processing content of the controller and an example of the display content of the screen in a case where the lumen is included in the central region of the frame;

FIG. 21 is a flowchart illustrating an example of a flow of a machine learning process;

FIG. 22 is a flowchart illustrating an example of a flow of a medical support process;

FIG. 23 is a conceptual diagram illustrating a modification example of the certainty information obtained in a case where the lumen is included in the central region of the frame;

FIG. 24 is a conceptual diagram illustrating a modification example of the certainty information obtained in a case where the lumen is included in a region other than the central region of the frame; and

FIG. 25 is a conceptual diagram illustrating an example of a series of processes in which a processor included in a computer gives a process execution request to an external device via a network, the external device executes a process corresponding to the process execution request, and the processor included in the computer receives a processing result from the external device.

### DETAILED DESCRIPTION

Hereinafter, examples of embodiments of a medical support device, an endoscope apparatus, a medical support method, and a program according to the present disclosure will be described with reference to the accompanying drawings.

First, the terms used in the following description will be described.

CPU refers to an abbreviation of “Central Processing Unit”. GPU is an abbreviation of “Graphics Processing Unit”. GPGPU is an abbreviation of “General-Purpose computing on Graphics Processing Units”. APU is an abbreviation of “Accelerated Processing Unit”. TPU is an abbreviation of a “Tensor Processing Unit”. RAM is an abbreviation of “Random Access Memory”. EEPROM is an abbreviation of “Electrically Erasable Programmable Read-Only Memory”. ASIC is an abbreviation of “Application Specific Integrated Circuit”. PLD is an abbreviation of “Programmable Logic Device”. FPGA is an abbreviation of “Field-Programmable Gate Array”. SoC is an abbreviation of “System-on-a-Chip”. SSD is an abbreviation of “Solid State Drive”. USB is an abbreviation of “Universal Serial Bus”. HDD is an abbreviation of “Hard Disk Drive”. EL is an abbreviation of “Electro-Luminescence”. CMOS is an abbreviation of “Complementary Metal Oxide Semiconductor”. CCD is an abbreviation of “Charge Coupled Device”. AI is an abbreviation of “Artificial Intelligence”. BLI is an abbreviation of “Blue Light Imaging”. LCI is an abbreviation of “Linked Color Imaging”. I/F is an abbreviation of “Interface”. SSL stands for “Sessile Serrated Lesion”. LAN is an abbreviation of “Local Area Network”. WAN is an abbreviation for “Wide Area Network”. 5G is an abbreviation for “5th Generation Mobile Communication System”.

In the following description, a processor with a reference numeral (hereinafter, simply referred to as a “processor”) may be one computing device or a combination of a plurality of computing devices. In addition, the processor may be one type of computing device or a combination of a plurality of types of computing devices. An example of the computing device is a CPU, a GPU, a GPGPU, an APU, or a TPU.

In the following description, a memory with a reference numeral is a memory, such as a RAM, temporarily storing information and is used as a work memory by the processor.

In the following description, a storage with a reference numeral is one or a plurality of non-volatile storage devices that store various programs, various parameters, and the like. An example of the non-volatile storage device is a flash memory, a magnetic disk, or a magnetic tape. In addition, another example of the storage is a cloud storage.

In the following embodiment, an external I/F with a reference numeral controls transmission and reception of various types of information between a plurality of devices connected to each other. An example of the external I/F is a USB interface. A communication I/F including a communication processor, an antenna, and the like may be applied to the external I/F. The communication I/F controls communication between a plurality of computers. An example of a communication standard applied to the communication I/F is a wireless communication standard including 5G, Wi-Fi (registered trademark), or Bluetooth (registered trademark).

In the following embodiment, “A and/or B” is synonymous with “at least one of A or B”. That is, “A and/or B” may mean only A, only B, or a combination of A and B. Further, in the present specification, the same concept as “A and/or B” is applied to a case where the connection of three or more matters is expressed by “and/or”.

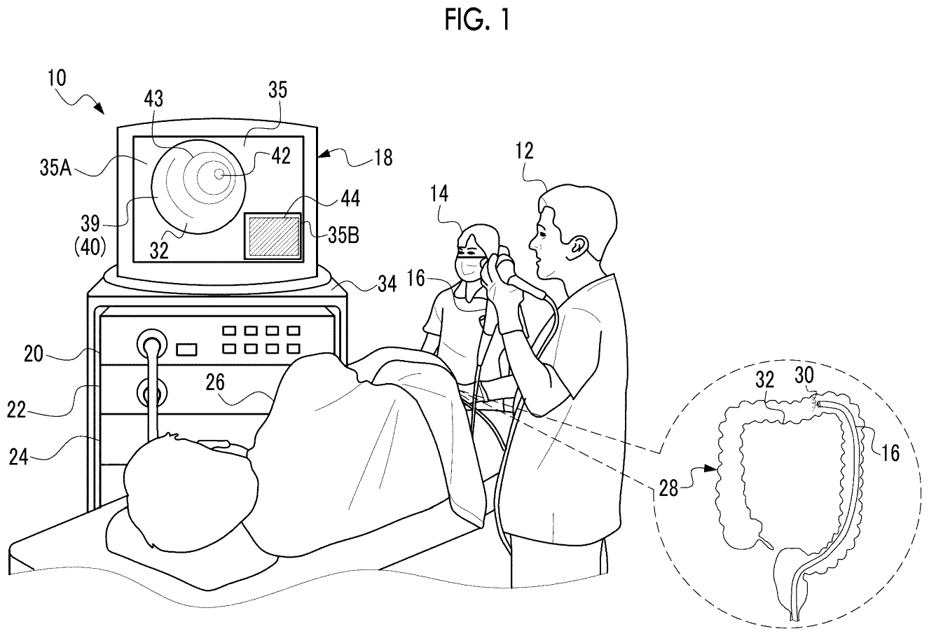

FIG. 1 is a conceptual diagram illustrating an example of an aspect in which an endoscope apparatus 10 is used. As illustrated in FIG. 1, the endoscope apparatus 10 is used by a doctor 12 in endoscopy or the like. The endoscopy is assisted by a staff member such as a nurse 14.

The endoscope apparatus 10 is connected to a communication device (not illustrated) such that it can communicate, and information obtained by the endoscope apparatus 10 is transmitted to the communication device. Examples of the communication device include a server, a personal computer, and/or a tablet terminal that manage various types of information such as electronic medical records. The communication device receives the information transmitted from the endoscope apparatus 10 and executes a process using the received information (for example, a process of storing the information in the electronic medical record or the like).

The endoscope apparatus 10 comprises an endoscope 16, a display device 18, a light source device 20, a control device 22, and a medical support device 24. In the present embodiment, the endoscope apparatus 10 is an example of an “endoscope apparatus” according to the present disclosure, and the medical support device 24 is an example of a “medical support device” according to the present disclosure.

The endoscope apparatus 10 is a modality for performing a medical examination on a large intestine 28, which is a luminal organ included in a body of a subject 26 (for example, a patient), using the endoscope 16. In the present embodiment, the large intestine 28 is an object to be observed by the doctor 12.

The endoscope 16 is used by the doctor 12 and is inserted into the body of the subject 26. In the present embodiment, the endoscope 16 is inserted into the large intestine 28 of the subject 26. In the present embodiment, the endoscope 16 is an example of an “endoscope” according to the present disclosure, and the large intestine 28 is an example of a “luminal organ” according to the present disclosure.

The endoscope apparatus 10 causes the endoscope 16 inserted into the large intestine 28 of the subject 26 to image the inside of the large intestine 28 including a lumen 42 and performs various medical treatments on the large intestine 28 as necessary. The large intestine 28 has the lumen 42. The endoscope 16 is inserted into the lumen 42. The position of the lumen 42 in the large intestine 28 can be medically specified on the basis of a form pattern of a plurality of folds 43 (for example, the shape, orientation, and the like of the plurality of folds 43) which are feature regions in the large intestine 28. In the present embodiment, the position of the lumen 42 is recognized by AI that has learned various types of information, such as the form pattern of the plurality of folds 43, using machine learning, and the recognition result is provided as visually ascertainable information to the doctor 12, which will be described in detail below. In the present embodiment, the lumen 42 is an example of a “lumen” according to the present disclosure.

The endoscope apparatus 10 images the inside of the large intestine 28 including the lumen 42, acquires an image showing an aspect including the lumen 42 in the large intestine 28, and outputs the acquired image. In the present embodiment, the endoscope apparatus 10 is an endoscope apparatus having an optical imaging function of irradiating the inside of the large intestine 28 with light 30 and capturing reflected light reflected by an intestinal wall 32 of the large intestine 28.

In addition, here, the endoscopy of the large intestine 28 is given as an example. However, this is only an example, and the present disclosure is applicable to the endoscopy of a luminal organ such as an esophagus, a stomach, a duodenum, or a trachea.

The light source device 20, the control device 22, and the medical support device 24 are installed in a wagon 34. A plurality of tables are provided in the wagon 34 along a vertical direction, and the medical support device 24, the control device 22, and the light source device 20 are installed from a lower table to an upper table. In addition, the display device 18 is installed on the uppermost table in the wagon 34.

The control device 22 controls the entire endoscope apparatus 10. The medical support device 24 performs various types of image processing on the image obtained by imaging the intestinal wall 32 with the endoscope 16 under the control of the control device 22.

The display device 18 displays various types of information including the image. An example of the display device 18 is a liquid crystal display or an EL display. In addition, a tablet terminal with a display may be used instead of the display device 18 or together with the display device 18.

A screen 35 is displayed on the display device 18. The screen 35 includes a plurality of display regions. The plurality of display regions are disposed side by side in the screen 35. In the example illustrated in FIG. 1, a first display region 35A and a second display region 35B are illustrated as an example of a plurality of display regions. A size of the first display region 35A is larger than a size of the second display region 35B. The first display region 35A is used as a main display region, and the second display region 35B is used as a sub-display region. A size relationship between the first display region 35A and the second display region 35B is not limited to this and may be any size relationship that falls within the screen 35.

An endoscope video image 39 is displayed in the first display region 35A. The endoscope video image 39 is a video image generated by imaging the inside of the large intestine 28 of the subject 26 with the endoscope 16. The intestinal wall 32 included in the endoscope video image 39 includes the lumen 42 as a region of interest (that is, a region to be observed) at which the doctor 12 gazes, and the doctor 12 can visually recognize the aspect of the intestinal wall 32 including the lumen 42 through the endoscope video image 39.

The image displayed in the first display region 35A is one frame 40 included in a video image configured to include a plurality of frames 40 arranged in time series. That is, the plurality of frames 40 arranged in time series are displayed in the first display region 35A at a predetermined frame rate (for example, a dozen frames/second or a few dozen frames/second).

An example of the video image displayed in the first display region 35A is a video image in a live view mode. The live view mode is only an example, and the video image may be a video image, such as a video image in a post view mode, that is temporarily stored in a memory or the like and then displayed. In addition, each frame included in a recording video image stored in the memory or the like may be reproduced and displayed as the endoscope video image 39 on the screen 35 (for example, in the first display region 35A).

The second display region 35B is displayed on the lower right side of the screen 35 in a front view. The second display region 35B may be displayed at any position in the screen 35 of the display device 18 and is preferably displayed at a position that can be contrasted with the endoscope video image 39. Auxiliary information 44 for assisting the doctor 12 in medical determination or the like is displayed in the second display region 35B. The auxiliary information 44 is information that is referred to by the doctor 12. Examples of the auxiliary information 44 include various types of information related to the subject 26 into which the endoscope 16 is inserted and/or various types of information obtained by performing a medical support process which will be described below.

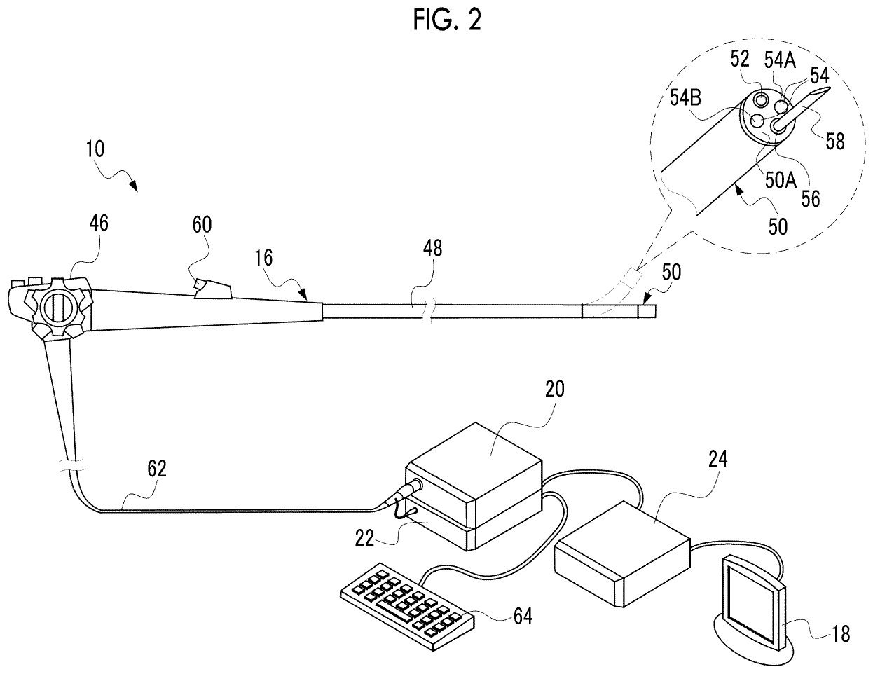

FIG. 2 is a conceptual diagram illustrating an example of an overall configuration of the endoscope apparatus 10. As illustrated in FIG. 2, the endoscope 16 comprises an operation unit 46 and an insertion portion 48. The insertion portion 48 is partially curved by the operation of the operation unit 46. The insertion portion 48 is inserted into the large intestine 28 while being curved according to the shape of the large intestine 28 (see FIG. 1) according to the operation of the operation unit 46 by the doctor 12 (see FIG. 1).

A camera 52, an illumination device 54, and a treatment tool opening 56 are provided in a distal end part 50 of the insertion portion 48. The camera 52 and the illumination device 54 are provided on a distal end surface 50A of the distal end part 50. In addition, here, the form in which the camera 52 and the illumination device 54 are provided on the distal end surface 50A of the distal end part 50 is given as an example. However, this is only an example. The camera 52 and the illumination device 54 may be provided on a side surface of the distal end part 50 such that the endoscope 16 is configured as a side-viewing endoscope.

The camera 52 is mounted on the endoscope 16 and is inserted into a body cavity of the subject 26 and images the region to be observed to generate the frame 40. In the present embodiment, the camera 52 images the inside of the large intestine 28 including the lumen 42 to generate the endoscope video image 39 including a plurality of frames 40 arranged in time series. An example of the camera 52 is a CMOS camera. However, this is only an example, and the camera 52 may be other types of cameras such as CCD cameras. In the present embodiment, the frame 40 is an example of a “medical image” and an “endoscope image” according to the present disclosure.

The illumination device 54 has illumination windows 54A and 54B. The illumination device 54 emits the light 30 (see FIG. 1) through the illumination windows 54A and 54B. Examples of the type of the light 30 emitted from the illumination device 54 include visible light (for example, white light) and invisible light (for example, near-infrared light). In addition, the illumination device 54 emits special light through the illumination windows 54A and 54B. Examples of the special light include light for BLI and/or light for LCI. The camera 52 images the inside of the large intestine 28 using an optical method in a state in which the illumination device 54 irradiates the inside of the large intestine 28 with the light 30.

The treatment tool opening 56 is an opening through which a treatment tool 58 protrudes from the distal end part 50. Further, the treatment tool opening 56 is also used as a suction port for sucking blood, body waste, and the like and a delivery port for sending out a fluid.

A treatment tool insertion opening 60 is formed in the operation unit 46, and the treatment tool 58 is inserted into the insertion portion 48 through the treatment tool insertion opening 60. The treatment tool 58 passes through the insertion portion 48 and protrudes from the treatment tool opening 56 to the outside. In the example illustrated in FIG. 2, an aspect in which, as the treatment tool 58, a puncture needle protrudes from the treatment tool opening 56 is illustrated. Here, the puncture needle is given as an example of the treatment tool 58. However, this is only an example. The treatment tool 58 may be grasping forceps, a papillotomy knife, a snare, a catheter, a guide wire, a cannula, and/or a puncture needle with a guide sheath.

The endoscope 16 is connected to the light source device 20 and the control device 22 through a universal cord 62. The medical support device 24 and a receiving device 64 are connected to the control device 22. In addition, the display device 18 is connected to the medical support device 24. That is, the control device 22 is connected to the display device 18 through the medical support device 24.

In addition, here, the medical support device 24 is given as an example of an external device for expanding the functions of the control device 22. Therefore, a form in which the control device 22 and the display device 18 are indirectly connected to each other through the medical support device 24 is given as an example. However, this is only an example. For example, the display device 18 may be directly connected to the control device 22. In this case, for example, the functions of the medical support device 24 may be provided in the control device 22, or the control device 22 may be provided with a function of directing a server (not illustrated) to execute the same process as the process (for example, a medical support process which will be described below) performed by the medical support device 24, receiving a result of the process by the server, and using the result.

The receiving device 64 receives an instruction from the doctor 12 and outputs the received instruction as an electric signal to the control device 22. Examples of the receiving device 64 include a keyboard, a mouse, a touch panel, a foot switch, a microphone, and/or a remote control device.

The control device 22 controls the light source device 20, transmits and receives various signals to and from the camera 52, or transmits and receives various signals to and from the medical support device 24.

The light source device 20 emits light and supplies the light to the illumination device 54 under the control of the control device 22. A light guide is provided in the illumination device 54, and the light supplied from the light source device 20 is emitted from the illumination windows 54A and 54B via the light guide. The control device 22 directs the camera 52 to perform imaging, acquires the endoscope video image 39 (see FIG. 1) from the camera 52, and outputs the endoscope video image 39 to a predetermined output destination (for example, the medical support device 24).

The medical support device 24 performs various types of image processing on the endoscope video image 39 input from the control device 22 to support a medical treatment (here, for example, endoscopy). The medical support device 24 outputs the endoscope video image 39 subjected to various types of image processing to a predetermined output destination (for example, the display device 18).

In addition, here, the form in which the endoscope video image 39 output from the control device 22 is output to the display device 18 through the medical support device 24 has been described as an example. However, this is only an example. For example, the control device 22 and the display device 18 may be connected to each other, and the endoscope video image 39 subjected to the image processing by the medical support device 24 may be displayed on the display device 18 through the control device 22.

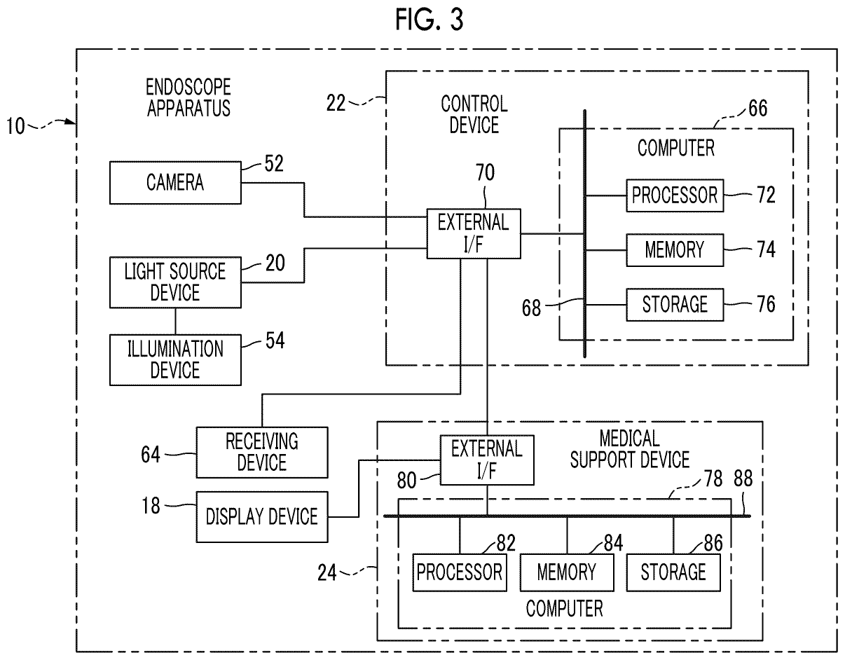

FIG. 3 is a block diagram illustrating an example of a hardware configuration of an electrical system of the endoscope apparatus 10. As illustrated in FIG. 3, the control device 22 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 22. The memory 74 and the storage 76 are used by the processor 72.

The external I/F 70 transmits and receives various types of information between one or more devices (hereinafter, also referred to as “first external devices”) outside the control device 22 and the processor 72.

As one of the first external devices, the camera 52 is connected to the external I/F 70, and the external I/F 70 transmits and receives various types of information between the camera 52 and the processor 72. The processor 72 controls the camera 52 via the external I/F 70. In addition, the processor 72 acquires the endoscope video image 39 (see FIG. 1) obtained by imaging the inside of the large intestine 28 (see FIG. 1) with the camera 52 via the external I/F 70.

As one of the first external devices, the light source device 20 is connected to the external I/F 70, and the external I/F 70 transmits and receives various types of information between the light source device 20 and the processor 72. The light source device 20 supplies light to the illumination device 54 under the control of the processor 72. The illumination device 54 performs irradiation with the light supplied from the light source device 20.

As one of the first external devices, the receiving device 64 is connected to the external I/F 70. The processor 72 acquires the instruction received by the receiving device 64 via the external I/F 70 and executes a process corresponding to the acquired instruction.

The medical support device 24 comprises a computer 78 and an external I/F 80. The computer 78 comprises a processor 82, a memory 84, and a storage 86. The processor 82, the memory 84, the storage 86, and the external I/F 80 are connected to a bus 88. In the present embodiment, the computer 78 is an example of a “computer” according to the present disclosure, and the processor 82 is an example of a “processor” according to the present disclosure.

Since a hardware configuration (that is, the processor 82, the memory 84, and the storage 86) of the computer 78 is basically the same as the hardware configuration of the computer 66, a description of the hardware configuration of the computer 78 will be omitted here.

The external I/F 80 transmits and receives various types of information between one or more devices (hereinafter, also referred to as “second external devices”) outside the medical support device 24 and the processor 82.

As one of the second external devices, the control device 22 is connected to the external I/F 80. In the example illustrated in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscope video image 39 (see FIG. 1) from the processor 72 of the control device 22 via the external I/Fs 70 and 80 and performs various types of image processing on the acquired endoscope video image 39.

As one of the second external devices, the display device 18 is connected to the external I/F 80. The processor 82 controls the display device 18 via the external I/F 80 such that various types of information (for example, the endoscope video image 39 subjected to various types of image processing) are displayed on the display device 18.

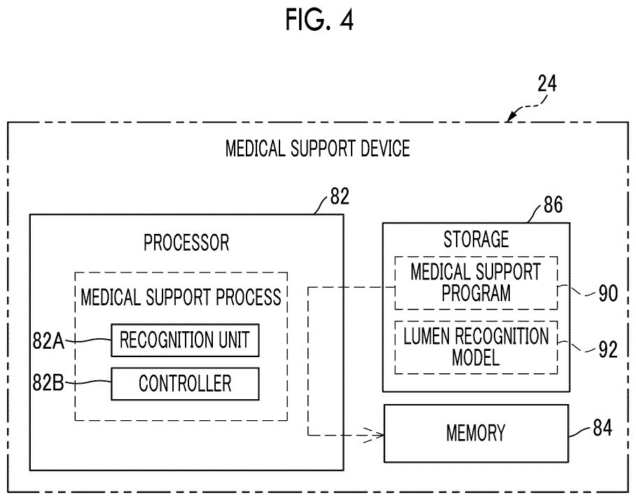

FIG. 4 is a block diagram illustrating an example of functions of main units of the processor 82 included in the medical support device 24 and an example of information stored in the storage 86. As illustrated in FIG. 4, the storage 86 stores a medical support program 90. The medical support program 90 is an example of a “program” according to the present disclosure. The processor 82 reads out the medical support program 90 from the storage 86 and executes the read-out medical support program 90 on the memory 84 to perform the medical support process. The processor 82 operates as a recognition unit 82A and a controller 82B according to the medical support program 90 executed on the memory 84 to implement the medical support process. In the present embodiment, the medical support process is an example of an “output process” according to the present disclosure.

A lumen recognition model 92 is stored in the storage 86. The lumen recognition model 92 is a trained model that is used in an AI-type process and is used by the recognition unit 82A, which will be described in detail below. In the present embodiment, the lumen recognition model 92 is an example of a “trained model” according to the present disclosure.

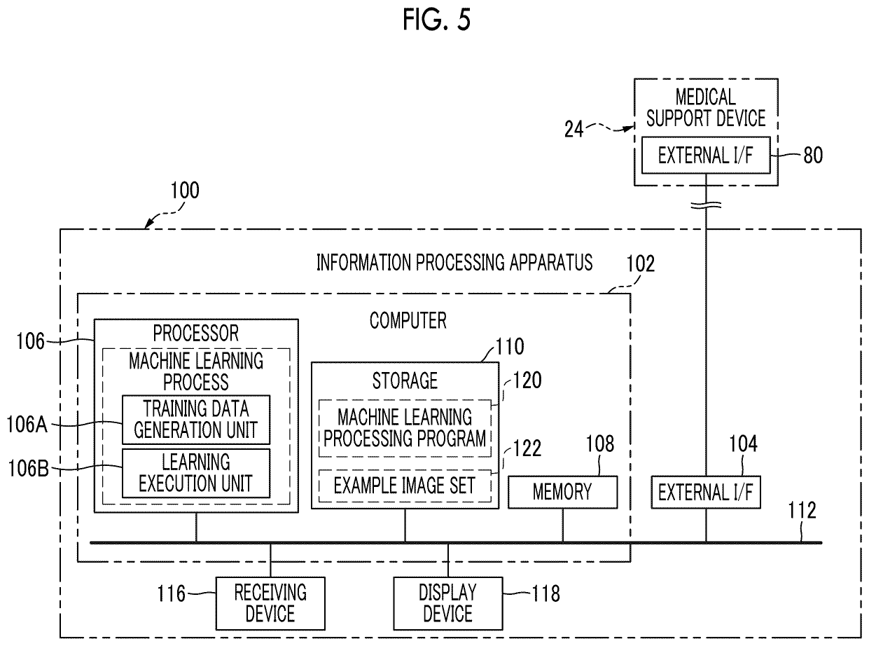

FIG. 5 is a block diagram illustrating an example of a hardware configuration of an electrical system of an information processing apparatus 100 used to generate the lumen recognition model 92. As illustrated in FIG. 5, the information processing apparatus 100 comprises a computer 102 and an external I/F 104. The computer 102 comprises a processor 106, a memory 108, and a storage 110. The processor 106, the memory 108, the storage 110, and the external I/F 104 are connected to a bus 112.

In addition, since a hardware configuration (that is, the processor 106, the memory 108, and the storage 110) of the computer 102 is basically the same as the hardware configuration of the computer 66, a description of the hardware configuration of the computer 102 will be omitted here.

The information processing apparatus 100 comprises a receiving device 116. The receiving device 116 is, for example, a keyboard and/or a mouse and receives an instruction from a user of the information processing apparatus 100 or the like. The receiving device 116 is connected to the bus 112. The processor 106 acquires the instruction received by the receiving device 116 and operates according to the acquired instruction.

A display device 118 displays various types of information including the image. An example of the display device 118 is a liquid crystal display or an EL display. The display device 118 is connected to the bus 112. The processor 106 displays the results obtained by executing various processes on the display device 118.

The external I/F 104 transmits and receives various types of information between one or more devices (hereinafter, also referred to as “third external devices”) outside the information processing apparatus 100 and the processor 106. As one of the third external devices, the medical support device 24 is connected to the external I/F 104. In the example illustrated in FIG. 5, the external I/F 80 of the medical support device 24 is connected to the external I/F 104. The external I/F 104 controls the transmission and reception of various types of information between the processor 82 (see FIGS. 3 and 4) of the medical support device 24 and the processor 106 of the information processing apparatus 100. For example, the information processing apparatus 100 generates the lumen recognition model 92 and transmits the generated lumen recognition model 92 to the medical support device 24 via the external I/Fs 80 and 104 in response to a request from the medical support device 24.

The storage 110 stores a machine learning processing program 120. The processor 106 reads out the machine learning processing program 120 from the storage 110 and executes the read-out machine learning processing program 120 on the memory 84 to perform a machine learning process. The processor 106 operates as a training data generation unit 106A and a learning execution unit 106B according to the machine learning processing program 120 executed on the memory 108 to implement the machine learning process.

The storage 110 stores an example image set 122. The example image set 122 is used by the training data generation unit 106A, which will be described in detail below.

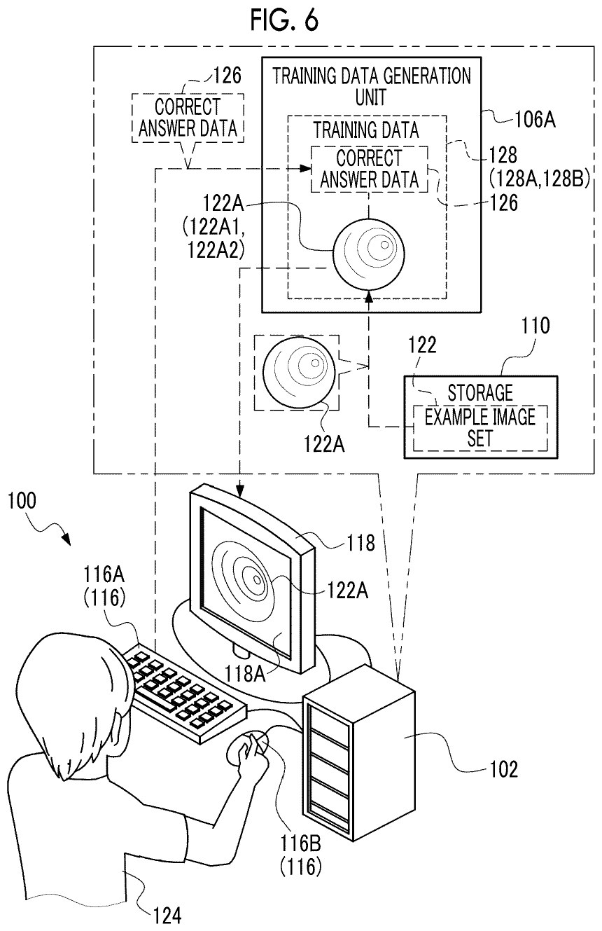

FIG. 6 is a conceptual diagram illustrating an example of the processing content of the training data generation unit 106A. As illustrated in FIG. 6, the information processing apparatus 100 is used by an annotator 124. The annotator 124 means a worker who adds annotations for machine learning to given data (that is, a worker who performs labeling).

In the example illustrated in FIG. 6, a keyboard 116A and a mouse 116B are illustrated as an example of the receiving device 116. The annotator 124 issues an instruction to the computer 102 via the keyboard 116A and the mouse 116B.

The example image set 122 includes a plurality of example images 122A having different contents. The example image 122A is an image determined in advance as a medical image used for an object recognition process (for example, a process in which the recognition unit 82A recognizes the lumen 42 on the basis of the frame 40 and the lumen recognition model 92). The image determined in advance as the medical image used for the object recognition process is an image corresponding to the frame 40. In other words, the image corresponding to the frame 40 can also be said to be an image assumed to be the frame 40. In other words, the image assumed to be the frame 40 can also be said to be an image indicating a sample of the frame 40. Here, a first example of the image indicating the sample of the frame 40 is an image obtained by actually imaging the inside of the large intestine with a camera. A second example of the image indicating the sample of the frame 40 is a virtually created image (for example, an image generated by generative AI, such as Stable Diffusion or Midjourney).

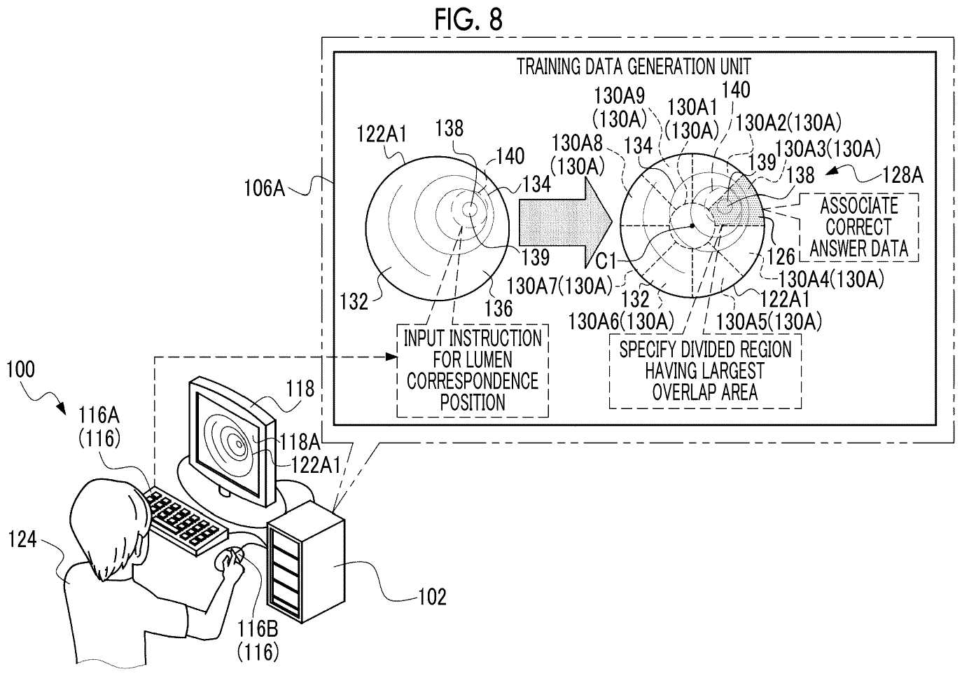

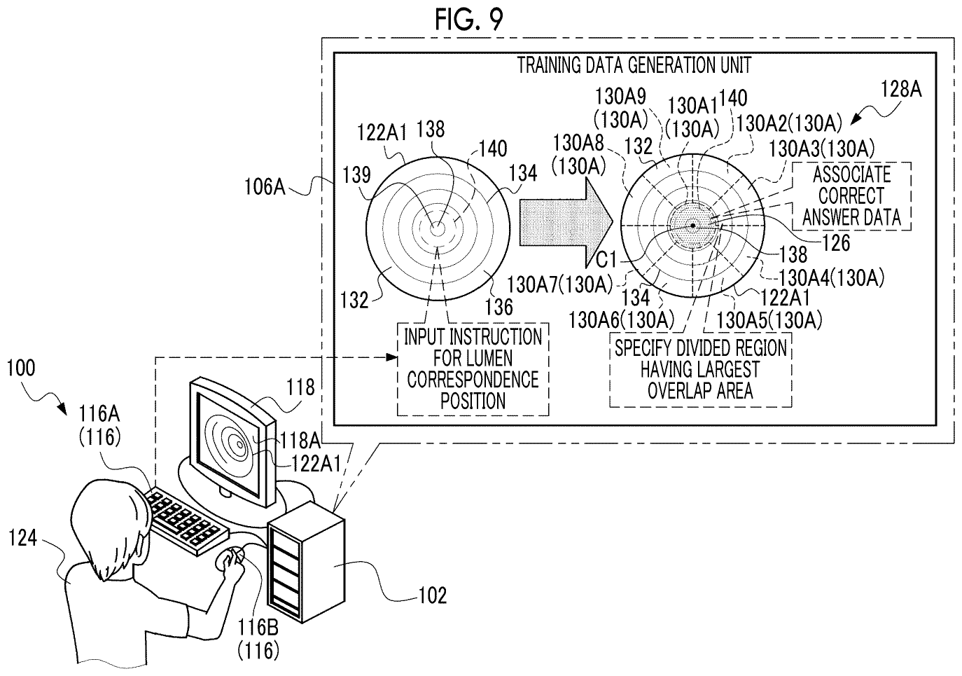

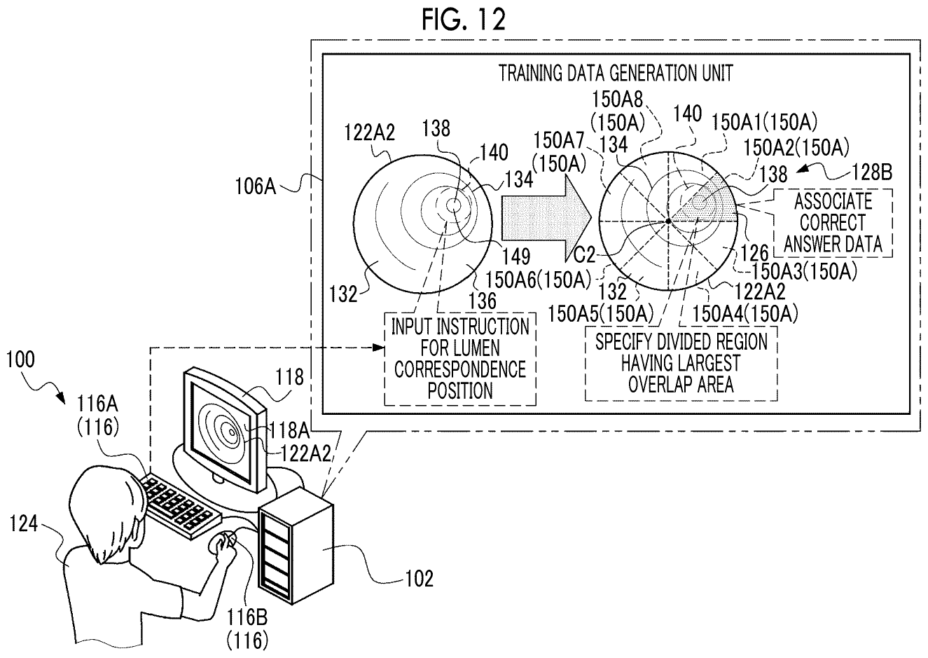

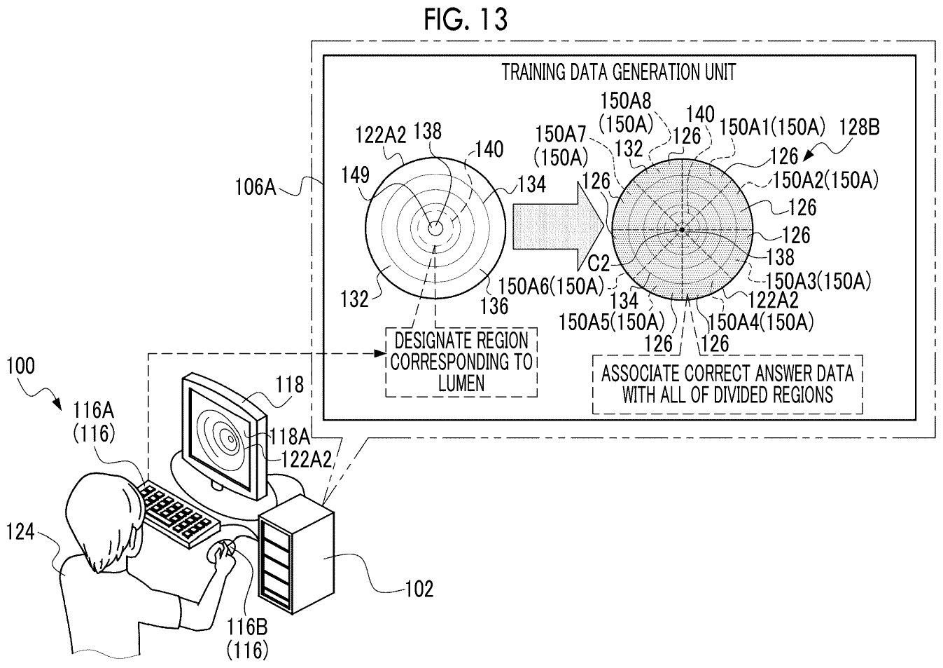

The training data generation unit 106A acquires the example image 122A from the example image set 122 in response to the instruction received by the receiving device 116. The training data generation unit 106A displays the example image 122A on a screen 118A of the display device 118. In a state in which the example image 122A is displayed on the screen 118A, the annotator 124 inputs an instruction for a lumen correspondence position, which is the position of the lumen included in the example image 122A in the example image 122A, to the training data generation unit 106A via the receiving device 116. The training data generation unit 106A associates correct answer data 126 (annotation data) with the example image 122A on the basis of the lumen correspondence position corresponding to the instruction through the receiving device 116 to generate training data 128. The association of the correct answer data 126 with the example image 122A is implemented by attaching an annotation capable of specifying the lumen correspondence position as the correct answer data 126 to the lumen correspondence position in the example image 122A. Examples of the lumen correspondence position include a lumen correspondence position 139 illustrated in FIGS. 8 and 9 and a lumen correspondence position 149 illustrated in FIGS. 12 and 13, which will be described in detail below.

In this way, the training data generation unit 106A repeatedly performs the process of associating the correct answer data 126 with each of the example images 122A included in the example image set 122 in response to the instruction given from the annotator 124 to generate a plurality of training data items 128.

The training data 128 is broadly divided into training data 128A as a comparative example for the present disclosure and training data 128B to which the present disclosure is applied. The training data 128A is data obtained by associating the correct answer data 126 with an example image 122A1 which is a first example of the example image 122A, and the training data 128B is data obtained by associating the correct answer data 126 with an example image 122A2 which is a second example of the example image 122A. Here, the training data 128B is an example of “training data” according to the present disclosure, and the training data 128A is a comparative example for the training data 128B. In addition, the example image 122A2 is an example of an “example image” according to the present disclosure, and the example image 122A1 is a comparative example for the example image 122A2. Further, the correct answer data 126 is an example of “correct answer data” according to the present disclosure.

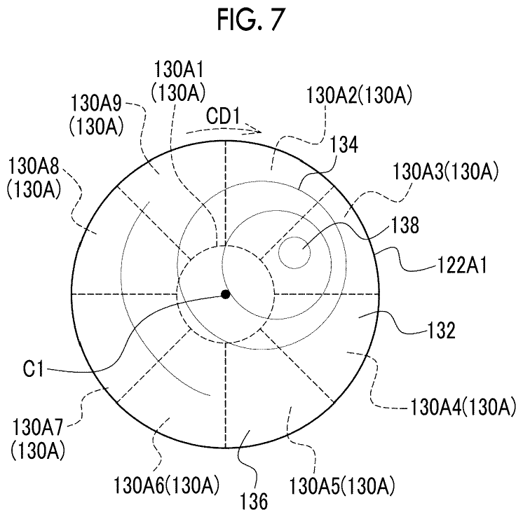

FIG. 7 is a conceptual diagram illustrating an example of a configuration of the example image 122A1. As illustrated in FIG. 7, a large intestine 132 is included in the example image 122A1. In the example illustrated in FIG. 7, an intestinal wall 136 in which a plurality of folds 134 are formed and a lumen 138 are included in the example image 122A1. Here, the lumen 138 is an example of a “sample of a lumen” according to the present disclosure.

The example image 122A1 is divided into a plurality of divided regions 130A. The plurality of divided regions 130A include a central region 130A1 and eight radial regions 130A2 to 130A9. The central region 130A1 is a circular region having a center matched with a center C1 of the example image 122A1. The radial regions 130A2 to 130A9 are regions that are present radially from the central region 130A1 toward an outer edge of the example image 122A1 and are disposed along a circumferential direction CD1 of the example image 122A1 (in other words, around the center C1 of the example image 122A1).

FIG. 8 is a conceptual diagram illustrating an example of a method in which the training data generation unit 106A associates the correct answer data 126 with the example image 122A1 to generate the training data 128A. As illustrated in FIG. 8, in a state in which the example image 122A1 is displayed on the screen 118A, the annotator 124 inputs an instruction for the lumen correspondence position 139, which is the position of the lumen 138 included in the example image 122A1 in the example image 122A1, to the training data generation unit 106A via the receiving device 116. The training data generation unit 106A displays a circular frame 140 to be superimposed on the example image 122A1 in response to the instruction received by the receiving device 116 and disposes the frame 140 at a position surrounding the lumen 138 included in the example image 122A1. The frame 140 is a mark that defines the lumen correspondence position 139 in the example image 122A1. That is, the position of a region surrounded by the frame 140 in the example image 122A1 is the lumen correspondence position 139. The size and position of the frame 140 are freely changed on the screen 118A in response to the instruction received by the receiving device 116. Here, the shape of the frame 140 is a circular shape, but may be a shape other than the circular shape. In addition, the size of the frame 140 can be changed in response to the instruction received by the receiving device 116.

The annotator 124 gives a confirmation instruction, which is an instruction to confirm the lumen correspondence position 139, to the training data generation unit 106A via the receiving device 116 in a state in which the frame 140 is disposed at the position surrounding the lumen 138. Then, the training data generation unit 106A confirms the lumen correspondence position 139.

The training data generation unit 106A specifies the divided region 130A having the largest overlap area with the frame 140 defining the lumen correspondence position 139 among the plurality of divided regions 130A. Then, the training data generation unit 106A associates the correct answer data 126 as an annotation capable of specifying the divided region 130A including the lumen 138 with the divided region 130A (in the example illustrated in FIG. 8, a radial region 130A3) specified from the plurality of divided regions 130A to generate the training data 128A.

Further, in the example illustrated in FIG. 8, an aspect in which the correct answer data 126 is associated with the radial region 130A3 is illustrated. However, this is only an example. For example, as illustrated in FIG. 9, in a case where the divided region 130A having the largest overlap area overlap with the frame 140 among the plurality of divided regions 130A is the central region 130A1, the training data generation unit 106A associates the correct answer data 126 with the central region 130A1 to generate the training data 128A.

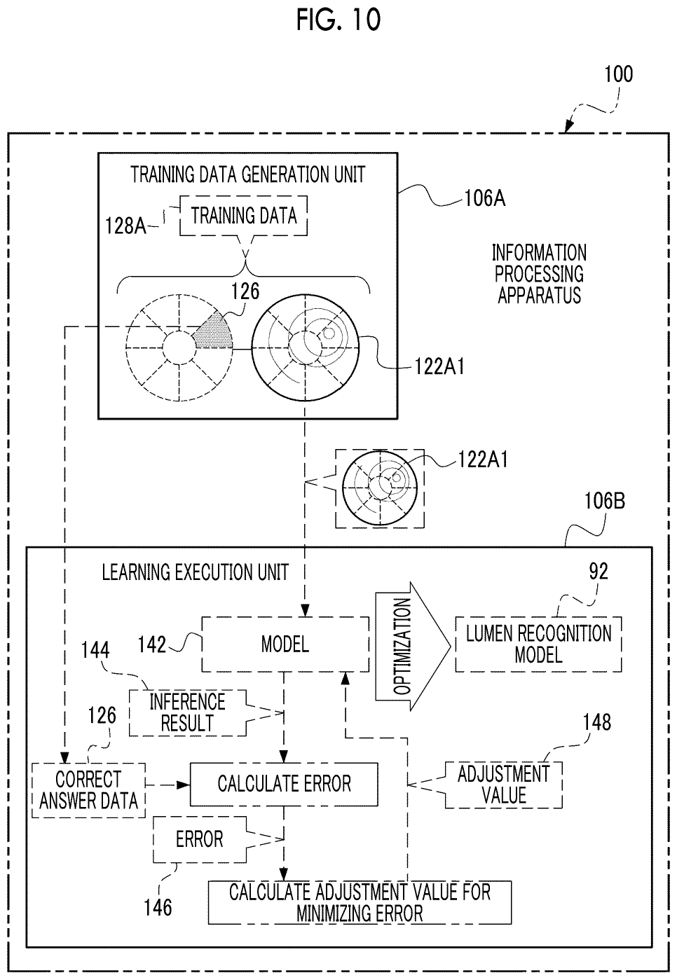

FIG. 10 is a conceptual diagram illustrating an example of an aspect in which the learning execution unit 106B executes machine learning using the training data 128A to generate the lumen recognition model 92. As illustrated in FIG. 10, in the information processing apparatus 100, the learning execution unit 106B acquires the training data 128A generated by the training data generation unit 106A. Then, the learning execution unit 106B executes machine learning using the training data 128A.

In the example illustrated in FIG. 10, the learning execution unit 106B includes a model 142. An example of the model 142 is a neural network. An example of the neural network is a convolutional neural network. The learning execution unit 106B inputs the example image 122A1 included in the training data 128A to the model 142. In a case where the example image 122A1 is input, the model 142 performs inference and outputs an inference result 144. The learning execution unit 106B calculates an error 146 between the inference result 144 and the correct answer data 126 included in the training data 128A.

The learning execution unit 106B calculates a plurality of adjustment values 148 for minimizing the error 146. Then, the learning execution unit 106B adjusts a plurality of optimization variables in the model 142 using the plurality of adjustment values 148 to optimize the model 142. For example, the plurality of optimization variables mean a plurality of connection weights and a plurality of offset values included in the model 142.

The learning execution unit 106B repeatedly performs the learning process of inputting the example image 122A1 to the model 142, calculating the error 146, calculating the plurality of adjustment values 148, and adjusting the plurality of optimization variables in the model 142 using the plurality of training data items 128A. That is, the learning execution unit 106B adjusts the plurality of optimization variables in the model 142 using the plurality of adjustment values 148 calculated such that the error 146 is minimized for each of the plurality of example images 122A1 included in the plurality of training data items 128A to optimize the model 142. The lumen recognition model 92 is generated by optimizing the model 142 in this way. The lumen recognition model 92 is transmitted from the information processing apparatus 100 to the medical support device 24 via the external I/Fs 80 and 104 (see FIG. 5) and is received by the medical support device 24. In the medical support device 24, the lumen recognition model 92 is stored in the storage 86 by the processor 82 (see FIG. 4). The lumen recognition model 92 stored in the storage 86 is used by the recognition unit 82A (see FIG. 4).

However, in a case where the lumen recognition model 92 is actually used, the frame 40 is input to the lumen recognition model 92. Then, the lumen recognition model 92 recognizes the lumen 42 (see FIG. 1) included in the input frame 40. However, the central region 130A1 (see FIGS. 7 to 9) of the example image 122A1 used in the training data 128A includes features other than the central region 130A1, that is, features of the radial regions 130A2 to 130A9 (see FIGS. 7 to 9). An example of the features of the radial regions 130A2 to 130A9 is a form pattern of a plurality of folds 134 (see FIGS. 7 to 9) in the large intestine 132.

As illustrated in FIGS. 7 to 9, since the example image 122A1 is classified into the central region 130A1 and the radial regions 130A2 to 130A9, information (for example, the form pattern of the plurality of folds 134 in the large intestine 132) useful for learning the presence of the lumen 138 is reduced by the central region 130A1 in the radial regions 130A2 to 130A9. Therefore, in a case where machine learning for causing the model 142 (FIG. 10) to recognize the lumen 138 (see FIGS. 7 to 9) is performed in a state in which the example image is classified into the central region 130A1 and the radial regions 130A2 to 130A9, the presence of the central region 130A1 classified for machine learning hinders machine learning for the regions other than the central region 130A1, that is, the radial regions 130A2 to 130A9.

There is a concern that the lumen recognition model 92 generated by performing machine learning for the radial regions 130A2 to 130A9, in which information (for example, the form pattern of the plurality of folds 134 in the large intestine 132) has been reduced by the presence of the central region 130A1 as described above, will recognize that the lumen 42 is included in the region other than the central region of the frame 40 even though the lumen 42 is not included in the region other than the central region of the frame 40 or will recognize that the lumen 42 is not included in the region other than the central region of the frame 40 even though the lumen 42 is included in the region other than the central region of the frame 40.

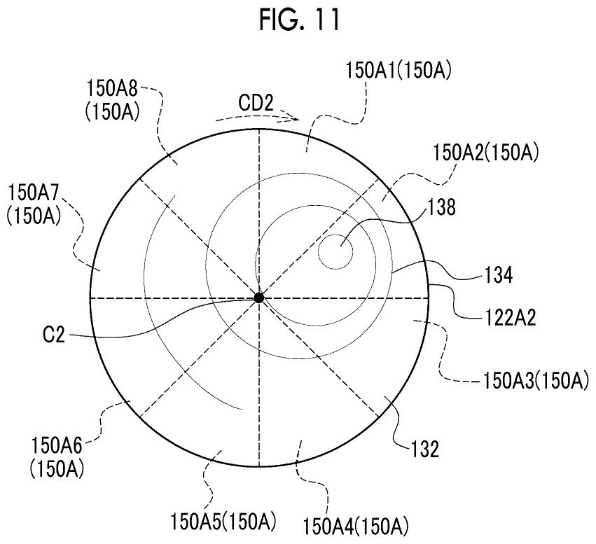

Therefore, in consideration of these circumstances, in the present embodiment, as illustrated in FIGS. 11 to 13, machine learning using the example image 122A2 instead of the example image 122A1 is performed on the model 142 to generate the lumen recognition model 92. Hereinafter, this will be described in detail.

FIG. 11 is a conceptual diagram illustrating an example of a configuration of the example image 122A2. As illustrated in FIG. 11, the example image 122A2 is different from the example image 122A1 (see FIGS. 7 to 9) in that it has a plurality of divided regions 150A instead of the plurality of divided regions 130A. The plurality of divided regions 150A are regions obtained by dividing the example image 122A2 along a circumferential direction CD2 (in other words, around a center C2 of the example image 122A2). In the example illustrated in FIG. 11, first to eighth divided regions 150A1 to 150A8 are given as an example of the plurality of divided regions 150A. The first to eighth divided regions 150A1 to 150A8 are eight regions obtained by dividing the example image 122A2 into eight equal parts along the circumferential direction CD2. In other words, the first to eighth divided regions 150A1 to 150A8 can also be said to be eight regions that are present radially from the center C2 of the example image 122A2 toward the outer edge of the example image 122A2. In the present embodiment, the first to eighth divided regions 150A1 to 150A8 are an example of a “plurality of regions” according to the present disclosure.

FIGS. 12 and 13 are conceptual diagrams illustrating an example of a method in which the training data generation unit 106A associates the correct answer data 126 with the example image 122A2 to generate the training data 128B. FIG. 12 illustrates an example of an aspect in which the correct answer data 126 is associated with the divided region 150A in a case where the lumen 138 is included at the same position as the lumen 138 illustrated in FIG. 8 in the example image 122A2. FIG. 13 illustrates an example of an aspect in which the correct answer data 126 is associated with the divided region 150A in a case where the lumen 138 is included at the same position as the lumen 138 illustrated in FIG. 9 in the example image 122A2.

As illustrated in FIG. 12, in a state in which the example image 122A2 is displayed on the screen 118A, the annotator 124 inputs an instruction for the lumen correspondence position 149, which is the position of the lumen 138 included in the example image 122A2 in the example image 122A2, to the training data generation unit 106A via the receiving device 116 in the same manner as in the example illustrated in FIGS. 8 and 9. The training data generation unit 106A displays the circular frame 140 to be superimposed on the example image 122A2 in response to the instruction received by the receiving device 116 and disposes the frame 140 at a position surrounding the lumen 138 included in the example image 122A2 in the same manner as in the example illustrated in FIGS. 8 and 9. The frame 140 is a mark that defines the lumen correspondence position 149 in the example image 122A2. That is, the position of the region surrounded by the frame 140 in the example image 122A2 is the lumen correspondence position 149.

The annotator 124 gives a confirmation instruction, which is an instruction to confirm the lumen correspondence position 149, to the training data generation unit 106A via the receiving device 116 in a state in which the frame 140 is disposed at the position surrounding the lumen 138. Then, the training data generation unit 106A confirms the lumen correspondence position 149.

The training data generation unit 106A specifies the divided region 150A having the largest overlap area with the frame 140 defining the lumen correspondence position 149 from the plurality of divided regions 150A in the same manner as in the example illustrated in FIGS. 8 and 9. Then, the training data generation unit 106A associates the correct answer data 126 as an annotation capable of specifying the divided region 150A including the lumen 138 with the divided region 150A (a second divided region 150A2 in the example illustrated in FIG. 12) specified from the plurality of divided regions 150A to generate the training data 128B.

On the other hand, as illustrated in FIG. 13, in a case where the lumen 138 is included in the central region (that is, a region corresponding to the central region 130A1 illustrated in FIG. 9) in the example image 122A, the training data generation unit 106A associates the correct answer data 126 with each of the divided regions 150A (that is, the first to eighth divided regions 150A1 to 150A8) to generate the training data 128B.

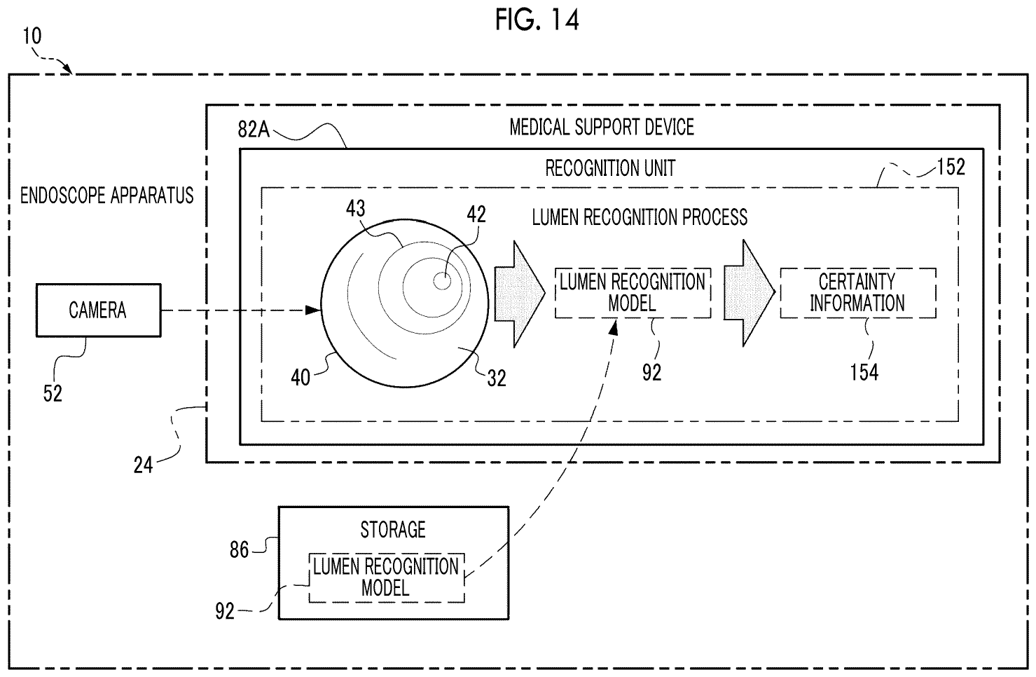

In the present embodiment, a plurality of training data items 128B (that is, a plurality of training data items 128B generated by using all of the example images 122A2 included in the example image set 122) generated by the training data generation unit 106A in this way are used for the machine learning by the learning execution unit 106B in the same manner as in the example illustrated in FIG. 10. The lumen recognition model 92 generated by performing the machine learning using the plurality of training data items 128B on the model 142 is used by the recognition unit 82A (see FIG. 14).

FIG. 14 is a conceptual diagram illustrating an example of a lumen recognition process 152 executed by the recognition unit 82A on the basis of the lumen recognition model 92 generated by performing the machine learning using the plurality of training data items 128B generated in the manner illustrated in FIGS. 12 and 13 on the model 142. As illustrated in FIG. 14, the recognition unit 82A executes the lumen recognition process 152 on the frame 40 generated by imaging the intestinal wall 32 having the lumen 42 in the large intestine 28 with the camera 52. The lumen recognition process 152 is a process of recognizing the lumen 42 included in the frame 40 using the lumen recognition model 92 stored in the storage 86 (in other words, a process of specifying the position of the lumen 42, which is included in the frame 40, in the frame 40 using the lumen recognition model 92). The recognition unit 82A acquires the frame 40 from the camera 52 and inputs the acquired frame 40 to the lumen recognition model 92 such that the lumen recognition model 92 generates certainty information 154. The certainty information 154 is an example of “certainty information” according to the present disclosure. Hereinafter, the certainty information 154 will be described in detail.

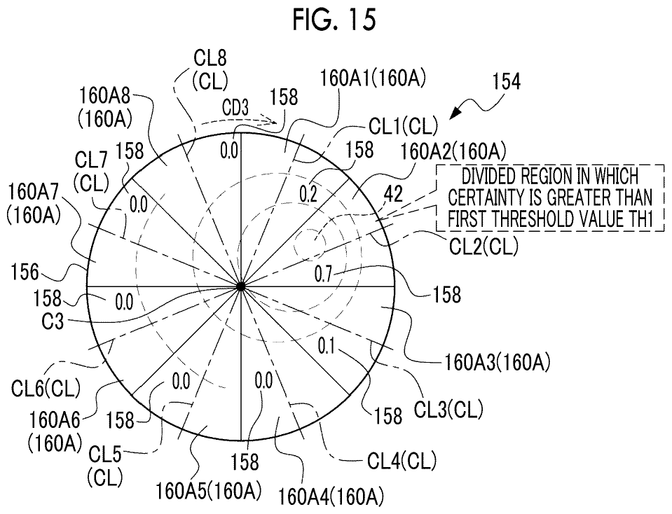

FIG. 15 is a conceptual diagram illustrating an example of a configuration of the certainty information 154 generated by the lumen recognition model 92 in a case where the lumen 42 is included in a region other than the central region of the frame 40. As illustrated in FIG. 15, the certainty information 154 is information in which a certainty 158 (for example, a probability that the lumen 138 will be present) is given to a map 156 corresponding to the frame 40. The map 156 is an example of an “image corresponding to a medical image” according to the present disclosure. In addition, here, the map 156 is given as an example. However, the frame 40 may be used instead of the map 156.

The map 156 has the same geometrical feature as the outer shape of the frame 40. That is, the map 156 can be said to be a circular image. The map 156 has a plurality of divided regions 160A corresponding to the plurality of divided regions 150A (see FIGS. 11 to 13). Each of the plurality of divided regions 160A is a region obtained by dividing the map 156 along a circumferential direction CD3 (in other words, around a center C3 of the map 156). In the example illustrated in FIG. 15, first to eighth divided regions 160A1 to 160A8 are given as an example of the plurality of divided regions 160A. The first to eighth divided regions 160A1 to 160A8 are eight regions obtained by dividing the map 156 into eight equal parts along the circumferential direction CD3. In other words, each of the first to eighth divided regions 160A1 to 160A8 can also be said to be a region obtained by radially dividing the map 156. In other words, the first to eighth divided regions 160A1 to 160A8 can also be said to be eight regions that are present radially from the center C3 of the map 156 toward an outer edge of the map 156. In the present embodiment, the first to eighth divided regions 160A1 to 160A8 are an example of a “plurality of divided regions” according to the present disclosure.

The map 156 is provided with a plurality of center lines CL. The plurality of center lines CL correspond to the plurality of divided regions 160A. Each of the plurality of center lines CLs is a virtual line that passes through a center of an arc of the corresponding divided region 160A from the center C3. In the example illustrated in FIG. 15, first to eighth center lines CL1 to CL8 are provided as an example of the plurality of center lines CL for the first to eighth divided regions 160A1 to 160A8. The first to eighth center lines CL1 to CL8 are disposed at intervals of 45 degrees around the center C3.

Each of the certainties 158 given to the plurality of divided regions 160A is compared with a first threshold value TH1 (for example, 0.4). Then, the divided region 160A to which a value exceeding the first threshold value TH1 is given as the certainty 158 is specified. The controller 82B performs the comparison between each certainty 158 and the first threshold value TH1 and the specification of the divided region 160A to which the value exceeding the first threshold value TH1 is given as the certainty 158. In the example illustrated in FIG. 15, the divided region 160A to which the value (0.7 in the example illustrated in FIG. 15) exceeding the first threshold value TH1 is given as the certainty 158 is the second divided region 160A2. The first threshold value TH1 may be a fixed value that is not changeable or may be a variable value that is changeable in response to the instruction or the like received by the receiving device 64. In the present embodiment, the first threshold value TH1 is an example of a “threshold value” according to the present disclosure, and the value exceeding the first threshold value TH1 is an example of a “value exceeding the threshold value” according to the present disclosure.

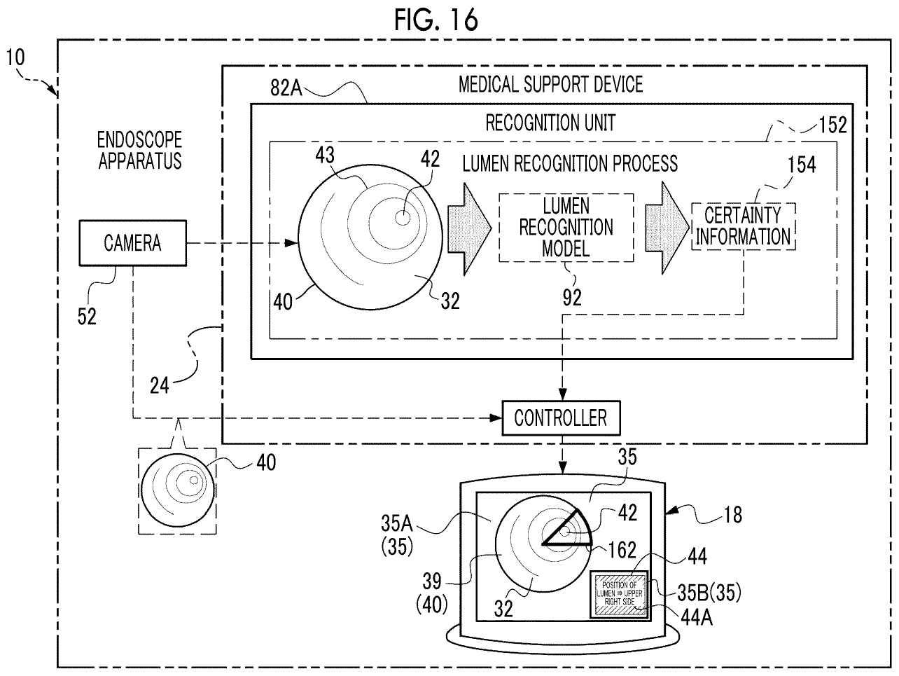

FIG. 16 is a conceptual diagram illustrating an example of the processing content of the controller 82B and an example of the display content of the screen 35 in a case where the lumen 42 is included in a region other than the central region of the frame 40. As illustrated in FIG. 16, the controller 82B acquires the frame 40 from the camera 52. The content included in the frame 40 acquired by the controller 82B is the same as the content included in the frame 40 input to the lumen recognition model 92 by the recognition unit 82A. The controller 82B displays the frame 40 acquired from the camera 52 in the first display region 35A.

Further, the controller 82B displays, in the first display region 35A, first visible information 162 as information indicating that the lumen 42 is present in any of the plurality of divided regions 160A on the basis of the certainty information 154. For example, in a case where the certainty information 154 is information in which the value exceeding the first threshold value TH1 is given as the certainty 158 to a single divided region 160A, the controller 82B determines that the lumen 42 is included in the region other than the central region of the frame 40 and displays the first visible information 162 in the first display region 35A.

In the example illustrated in FIG. 16, the first visible information 162 displayed in the first display region 35A is information capable of visually specifying that the first divided region 160A to which the value exceeding the first threshold value TH1 is given as the certainty 158 is the second divided region 160A2. In the example illustrated in FIG. 16, a mark that borders the outer periphery of an image region (a fan-shaped image region in the example illustrated in FIG. 16) corresponding to the second divided region 160A2 among all of the image regions of the frame 40 is used as the first visible information 162. Since the lumen 42 is included in the image region corresponding to the second divided region 160A2 in the frame 40, the first visible information 162 displayed in the first display region 35A can be said to be information capable of visually specifying the position of the lumen 42 included in the frame 40. In addition, the first visible information 162 is displayed in the frame 40 displayed in the first display region 35A. In the example illustrated in FIG. 16, an aspect in which the first visible information 162 is displayed to be superimposed on the frame 40 is illustrated.

In the example illustrated in FIG. 16, the controller 82B further displays text 44A as one of the auxiliary information items 44 in the second display region 35B. The text 44A is text indicating the position of the lumen 42 in the frame 40 displayed in the first display region 35A and is displayed in the second display region 35B in a case where the lumen 42 is included in a region other than the central region of the frame 40. In the example illustrated in FIG. 16, as the text 44A, text indicating that the lumen 42 is located on the upper right side of the frame 40 is illustrated.

In the present embodiment, the first visible information 162 and the text 44A are examples of “first information” and “visible information” according to the present disclosure.

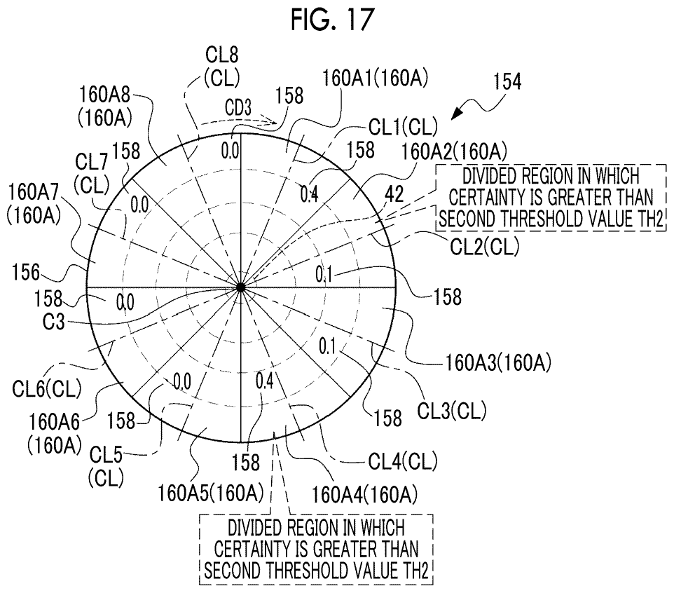

FIG. 17 is a conceptual diagram illustrating a first example of a configuration of the certainty information 154 generated by the lumen recognition model 92 in a case where the lumen 42 is included in the central region of the frame 40. As illustrated in FIG. 17, each of the certainties 158 given to the plurality of divided regions 160A is compared with a second threshold value TH2 (for example, 0.3). Then, the divided region 160A to which a value exceeding the second threshold value TH2 is given as the certainty 158 is specified. The controller 82B performs the comparison between each certainty 158 and the second threshold value TH2 and the specification of the divided region 160A to which the value exceeding the second threshold value TH2 is given as the certainty 158.

In the example illustrated in FIG. 17, the divided regions 160A to which the value (0.4 in the example illustrated in FIG. 17) exceeding the second threshold value TH2 is given as the certainty 158 are the first divided region 160A1 and the fourth divided region 160A4. A positional relationship between the first divided region 160A1 and the fourth divided region 160A4 to which the value exceeding the second threshold value TH2 is given as the certainty 158 is a positional relationship exceeding 90 degrees in the circumferential direction CD3 of the map 156 among the plurality of divided regions 160A. The positional relationship exceeding 90 degrees in the circumferential direction CD3 of the map 156 among the plurality of divided regions 160A is synonymous with a positional relationship in which an angle formed between two center lines CL exceeds 90 degrees. In the example illustrated in FIG. 17, the angle formed between the center line CL1 of the first divided region 160A1 and the center line CL4 of the fourth divided region 160A4 is 135 degrees and exceeds 90 degrees. Therefore, it can be said that the positional relationship between the first divided region 160A1 and the fourth divided region 160A4 is the positional relationship exceeding 90 degrees in the circumferential direction CD3 of the map 156 among the plurality of divided regions 160A.

In addition, the second threshold value TH2 used for comparison with each certainty 158 may be a fixed value that is not changeable or may be a variable value that is changeable in response to the instruction or the like received by the receiving device 64. In the present embodiment, the second threshold value TH2 is an example of a “threshold value” according to the present disclosure, and the value exceeding the second threshold value TH2 is an example of a “value exceeding the threshold value” according to the present disclosure.

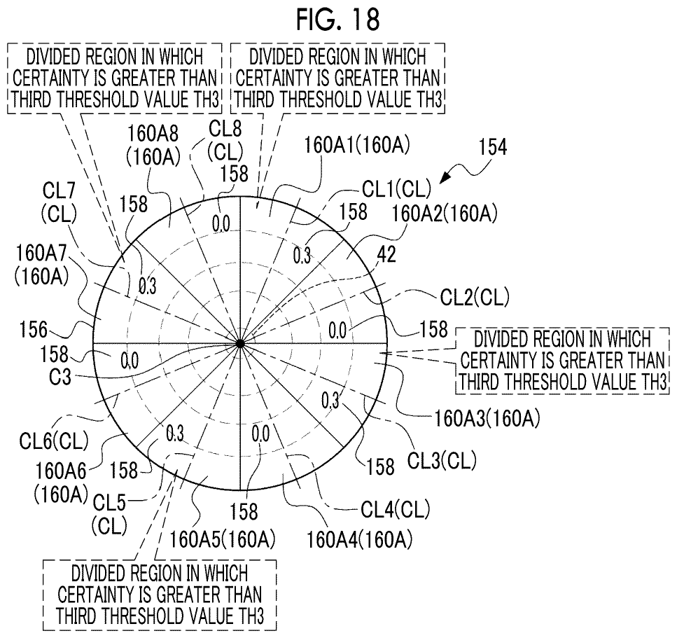

FIG. 18 is a conceptual diagram illustrating a second example of the configuration of the certainty information 154 generated by the lumen recognition model 92 in a case where the lumen 42 is included in the central region of the frame 40. As illustrated in FIG. 18, each of the certainties 158 given to the plurality of divided regions 160A is compared with a third threshold value TH3 (for example, 0.2). Then, the divided region 160A to which a value exceeding the third threshold value TH3 is given as the certainty 158 is specified. The controller 82B performs the comparison between each certainty 158 and the third threshold value TH3 and the specification of the divided region 160A to which the value exceeding the third threshold value TH3 is given as the certainty 158.

In the example illustrated in FIG. 18, the divided regions 160A to which the value (0.3 in the example illustrated in FIG. 18) exceeding the third threshold value TH3 is given as the certainty 158 are the first divided region 160A1, the third divided region 160A3, the fifth divided region 160A5, and the seventh divided region 160A7. The first divided region 160A1, the third divided region 160A3, the fifth divided region 160A5, and the seventh divided region 160A7 are four divided regions 160A that are disposed at regular intervals around the entire circumference of the map 156. In the example illustrated in FIG. 18, the regular interval around the entire circumference of the map 156 means an interval at which the angle formed between the center lines CL of the divided regions 160A is 90 degrees.

The third threshold value TH3 used for comparison with each certainty 158 may be a fixed value that is not changeable or may be a variable value that is changeable in response to the instruction or the like received by the receiving device 64. In the present embodiment, the third threshold value TH3 is an example of a “threshold value” according to the present disclosure, and the value exceeding the third threshold value TH3 is an example of a “value exceeding the threshold value” according to the present disclosure.

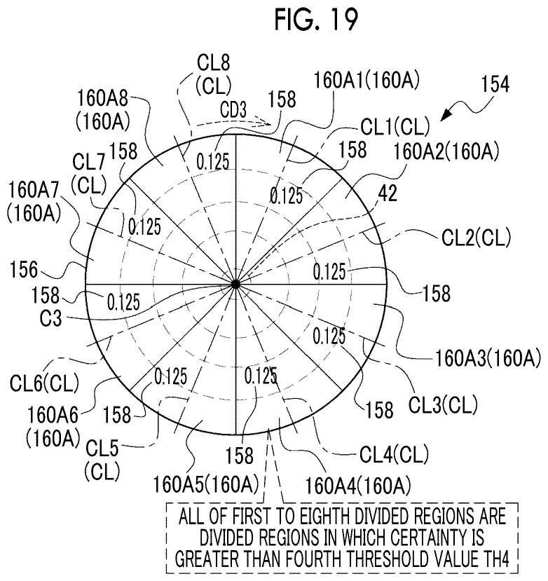

FIG. 19 is a conceptual diagram illustrating a third example of the configuration of the certainty information 154 generated by the lumen recognition model 92 in a case where the lumen 42 is included in the central region of the frame 40. As illustrated in FIG. 19, each of the certainties 158 given to the plurality of divided regions 160A is compared with a fourth threshold value TH4 (for example, 0.1). Then, the divided region 160A to which a value exceeding the fourth threshold value TH4 is given as the certainty 158 is specified. The controller 82B performs the comparison between each certainty 158 and the fourth threshold value TH4 and the specification of the divided region 160A to which the value exceeding the fourth threshold value TH4 is given as the certainty 158.

In the example illustrated in FIG. 19, the divided regions 160A to which the value (0.125 in the example illustrated in FIG. 19) exceeding the fourth threshold value TH4 is given as the certainty 158 are the first to eighth divided regions 160A1 to 160A8 (that is, all of the divided regions 160A).

The fourth threshold value TH4 used for comparison with each certainty 158 may be a fixed value that is not changeable or may be a variable value that is changeable in response to the instruction or the like received by the receiving device 64. In the present embodiment, the fourth threshold value TH4 is an example of a “threshold value” according to the present disclosure, and the value exceeding the fourth threshold value TH4 is an example of a “value exceeding the threshold value” according to the present disclosure.

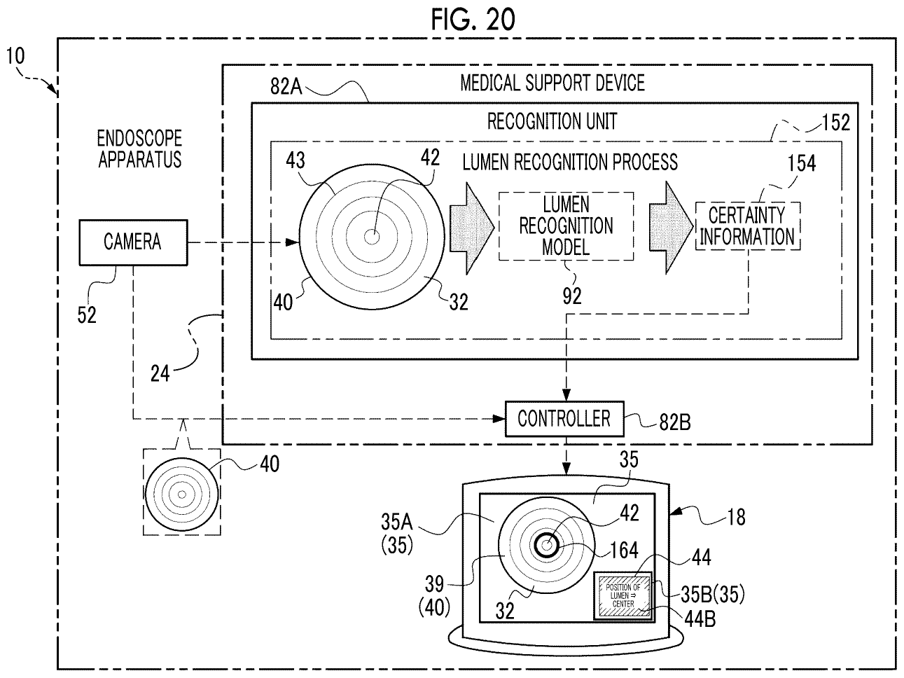

FIG. 20 is a conceptual diagram illustrating an example of the processing content of the controller 82B and an example of the display content of the screen 35 in a case where the lumen 42 is included in the central region of the frame 40. As illustrated in FIG. 20, the controller 82B displays the frame 40 in the first display region 35A in the same manner as in the example illustrated in FIG. 16.

In addition, in a case where the certainty information 154 is information in which the value exceeding the second threshold value TH2 is given as the certainty 158 to two or more divided regions 160A having a positional relationship exceeding 90 degrees in the circumferential direction CD3 of the map 156 among all of the divided regions 160A, the controller 82B determines that the lumen 42 is included in the central region of the frame 40 and displays second visible information 164 as information indicating that the lumen 42 is present in the central region of the frame 40 in the first display region 35A. Here, the certainty information 154 illustrated in FIG. 17 is given as an example of the information in which the value exceeding the second threshold value TH2 is given as the certainty 158 to two or more divided regions 160A having the positional relationship exceeding 90 degrees in the circumferential direction CD3 of the map 156 among all of the divided regions 160A.

In addition, in a case where the certainty information 154 is information in which the value exceeding the third threshold value TH3 is given as the certainty 158 to two or more divided regions 160A disposed at regular intervals around the entire circumference of the map 156 among all of the divided regions 160A, the controller 82B determines that the lumen 42 is included in the central region of the frame 40 and displays the second visible information 164 in the first display region 35A. Here, the certainty information 154 illustrated in FIG. 18 is given as an example of the information in which the value exceeding the third threshold value TH3 is given as the certainty 158 to two or more divided regions 160A disposed at regular intervals around the entire circumference of the map 156 among all of the divided regions 160A.

In addition, in a case where the certainty information 154 is information in which the value exceeding the fourth threshold value TH4 is given as the certainty 158 to all of the divided regions 160A, the controller 82B determines that the lumen 42 is included in the central region of the frame 40 and displays the second visible information 164 in the first display region 35A. Here, the certainty information 154 illustrated in FIG. 19 is an example of the information in which the value exceeding the fourth threshold value TH4 is given as the certainty 158 to all of the divided regions 160A.

The second visible information 164 displayed in the first display region 35A is information capable of visually specifying that the lumen 42 is present in the central region of the frame 40. In the example illustrated in FIG. 20, a mark that surrounds the outer periphery of the central region (for example, an image region corresponding to the central region 130A1 illustrated in FIG. 9) among all of the image regions of the frame 40 is used as the second visible information 164. Since the lumen 42 is included in the central region among all of the image regions of the frame 40, the second visible information 164 displayed in the first display region 35A can be said to be information capable of visually specifying the position of the lumen 42 included in the frame 40. Further, the second visible information 164 is displayed in the frame 40 that is displayed in the first display region 35A. In the example illustrated in FIG. 20, an aspect in which the second visible information 164 is displayed to be superimposed on the frame 40 is illustrated.

In the example illustrated in FIG. 20, the controller 82B further displays text 44B as one of the auxiliary information items 44 in the second display region 35B. The text 44B is displayed in the second display region 35B in a case where the lumen 42 is included in the central region of the frame 40. The text 44B is text indicating that the lumen 42 is included in the central region of the frame 40 displayed in the first display region 35A.

Further, in the present embodiment, the second visible information 164 and the text 44B are examples of “second information” and “visible information” according to the present disclosure.

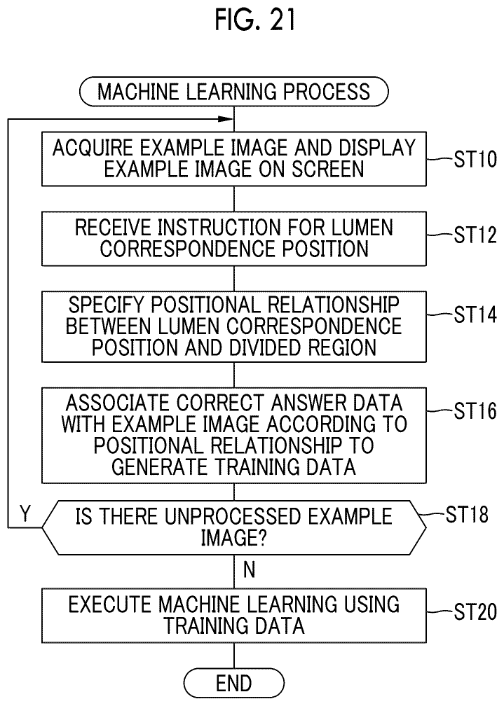

Next, an operation of the information processing apparatus 100 will be described with reference to FIG. 21.

In a machine learning process illustrated in FIG. 21, first, in Step ST10, the training data generation unit 106A acquires an unprocessed example image 122A2 from the example image set 122 stored in the storage 110. Here, the unprocessed example image 122A2 means the example image 122A2 that has not yet been used for the machine learning process. The training data generation unit 106A displays the example image 122A2 acquired from the example image set 122 on the screen 118A. After the process in Step ST10 is executed, the machine learning process proceeds to Step ST12.

In Step ST12, the training data generation unit 106A receives an instruction for the lumen correspondence position 149. After the process in Step ST12 is executed, the machine learning process proceeds to Step ST14.

In Step ST14, the training data generation unit 106A specifies the positional relationship between the lumen correspondence position 149 received in Step ST12 and the plurality of divided regions 150A. After the process in Step ST14 is executed, the machine learning process proceeds to Step ST16.

In Step ST16, the training data generation unit 106A associates the correct answer data 126 with the example image 122A2 acquired in Step ST10 according to the positional relationship specified in Step ST14. For example, in a case where the lumen correspondence position 149 is present in a region other than the central region of the example image 122A2, the correct answer data 126 is associated with the divided region 150A having the largest overlap area with the lumen correspondence position 149 in response to the instruction given from the annotator 124. In addition, for example, in a case where the lumen correspondence position 149 is present in the central region of the example image 122A2, the correct answer data 126 is associated with each of the divided regions 150A in response to the instruction given from the annotator 124. As described above, the training data generation unit 106A associates the correct answer data 126 with the example image 122A2 to generate the training data 128B. The training data 128B generated in this way is stored in a predetermined storage medium (for example, the storage 110). After the process in Step ST16 is executed, the machine learning process proceeds to Step ST18.

In Step ST18, the training data generation unit 106A determines whether or not the unprocessed example image 122A2 is present. In Step ST18, in a case where the unprocessed example image 122A2 is present, the determination result is “Yes”, and the machine learning process proceeds to Step ST10. In Step ST18, in a case where the unprocessed example image 122A2 is not present, the determination result is “No”, and the machine learning process proceeds to Step ST20.

In Step ST20, the learning execution unit 106B executes machine learning using a plurality of training data items 128B obtained by repeatedly executing the processes in Steps ST10 to ST18 to generate the lumen recognition model 92 (see FIG. 10). The lumen recognition model 92 is stored in the storage 86 of the medical support device 24 (see FIG. 4). After the process in Step ST20 is executed, the machine learning process is ended.

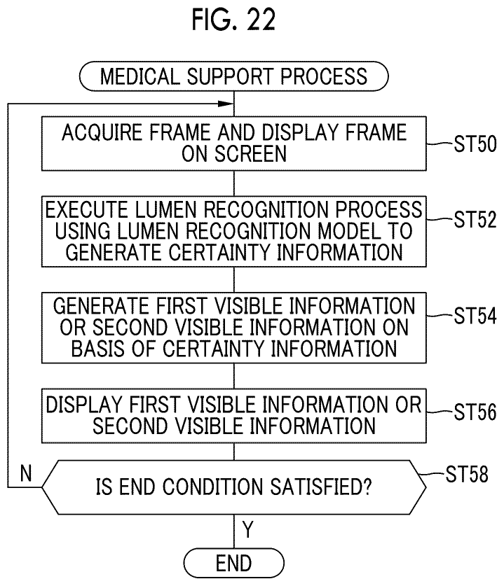

Next, an operation of a portion of the endoscope apparatus 10 according to the present disclosure will be described with reference to FIG. 22. A flow of a medical support process illustrated in FIG. 22 is an example of a “medical support method” according to the present disclosure.

In the medical support process illustrated in FIG. 22, in Step ST50, the recognition unit 82A and the controller 82B acquire the frame 40 from the camera 52. The controller 82B displays the frame 40 acquired from the camera 52 in the first display region 35A. After the process in Step ST50 is executed, the medical support process proceeds to Step ST52.

In Step ST52, the recognition unit 82A executes the lumen recognition process 152 using the lumen recognition model 92 stored in the storage 86 on the frame 40 acquired in Step ST50 to generate the certainty information 154. After the process in Step ST52 is executed, the medical support process proceeds to Step ST54.

In Step ST54, the controller 82B generates the first visible information 162 or the second visible information 164 on the basis of the certainty information 154 generated in Step ST52. For example, in a case where the certainty information 154 (that is, information indicating a distribution of the certainty 158 given to the map 156) is the information of the type illustrated in FIG. 15 (that is, in a case where the certainty information 154 is the information in which the value exceeding the first threshold value TH1 is given as the certainty 158 to a single divided region 160A), the first visible information 162 is generated. In addition, for example, in a case where the certainty information 154 is the information of the type illustrated in FIGS. 17 and 18, the second visible information 164 is generated. After the process in Step ST54 is executed, the medical support process proceeds to Step ST56.

In Step ST56, the controller 82B displays the first visible information 162 or the second visible information 164 generated in Step ST54 on the screen 35 (see FIGS. 16 and 20). After the process in Step ST56 is executed, the medical support process proceeds to Step ST58.

In Step ST58, the controller 82B determines whether or not a medical support process end condition is satisfied. An example of the medical support process end condition is a condition that an instruction to end the medical support process is given to the endoscope apparatus 10 (for example, a condition that the receiving device 64 receives the instruction to end the medical support process).

In a case where the medical support process end condition is not satisfied in Step ST58, the determination result is “No”, and the medical support process proceeds to Step ST50. In a case where the medical support process end condition is satisfied in Step ST58, the determination result is “Yes”, and the medical support process is ended.

In this manner, in the medical support process, the first visible information 162 is displayed on the screen 35 in a case in which the certainty information 154 (that is, information indicating a distribution of the certainty 158 given to the map 156) is the type of information illustrated in FIG. 15 (that is, in a case in which the certainty information 154 is information in which a value exceeding the first threshold value TH1 is given as the certainty 158 to a single divided region 160A), and the second visible information 164 is displayed on the screen 35 in a case in which the certainty information 154 is the type of information illustrated in FIGS. 17 and 18. In other words, the medical support process is a process capable of distinguishing between a case where the certainty information 154 is the type of information illustrated in FIG. 15 and a case where the certainty information 154 is the type of information illustrated in FIGS. 17 and 18.

As described above, in the present embodiment, the certainty information 154 is generated by inputting the frame 40 to the lumen recognition model 92. The certainty information 154 is information in which the certainty 158 of the lumen 42 being present in each of the first to eighth divided regions 160A1 to 160A8 obtained by dividing the map 156 along the circumferential direction CD3 is given to the first to eighth divided regions 160A1 to 160A8. In the present embodiment, in a case where the lumen 42 is included in a region other than the central region of the frame 40, the first visible information 162 is displayed in the first display region 35A as information indicating that the lumen 42 is present in any of the divided regions 160A on the basis of the certainty information 154. Further, in a case where the lumen 42 is included in the central region of the frame 40, the second visible information 164 is displayed in the first display region 35A as information indicating that the lumen 42 is included in the central region of the frame 40.

However, in the comparative example illustrated in FIGS. 7 to 9, the example image 122A1 is included in the training data 128A used for machine learning for generating the lumen recognition model 92. The training data 128A illustrated in FIGS. 8 and 9 as a comparative example for the training data 128B according to the present embodiment is generated by associating the correct answer data 126 with the example image 122A1 illustrated in FIG. 7 as a comparative example for the example image 122A2.

However, the central region 130A1 of the example image 122A1 also includes the features of the radial regions 130A2 to 130A9 (for example, the form pattern of the folds 134 included in each of the radial regions 130A2 to 130A9). That is, since the example image 122A1 is classified into the central region 130A1 and the radial regions 130A2 to 130A9, the information for learning the presence of the lumen 138 is reduced by the central region 130A1 in the radial regions 130A2 to 130A9. This means that the presence of the central region 130A1 hinders machine learning for the radial regions 130A2 to 130A9. There is a concern that the lumen recognition model 92 generated by the machine learning will recognize that the lumen 42 is included in the region other than the central region of the frame 40 even though the lumen 42 is not included in the region other than the central region of the frame 40 or will recognize that the lumen 42 is not included in the region other than the central region of the frame 40 even though the lumen 42 is included in the region other than the central region of the frame 40.

Therefore, in the present embodiment, the example image 122A2 included in the training data 128B used to generate the lumen recognition model 92 is not provided with the region corresponding to the central region 130A1. The example image 122A2 is divided into the first to eighth divided regions 150A1 to 150A8. Since the first to eighth divided regions 150A1 to 150A8 also include the information (for example, the form pattern of the folds 134) included in the central region 130A1 of the example image 122A1 given as the comparative example, machine learning with higher accuracy than the machine learning for the radial regions 130A2 to 130A9 can be performed on the first to eighth divided regions 150A1 to 150A8.

In the present embodiment, in a case where the lumen 138 is included in the central region of the example image 122A2, the correct answer data 126 is associated with all of the first to eighth divided regions 150A1 to 150A8. In a case where the lumen 138 is included in a region other than the central region of the example image 122A2, the correct answer data 126 is associated with the divided region 150A having the largest overlap area with the lumen 138 to generate the training data 128B. In the present embodiment, the position of the lumen 42, which is included in the frame 40, in the frame 40 is recognized by the lumen recognition model 92 obtained by the machine learning using the training data 128B generated in this way. Therefore, according to the present embodiment, the position of the lumen 42, which is included in the frame 40, in the frame 40 can be recognized by the lumen recognition model 92 with higher accuracy, as compared to a case where the position of the lumen 42, which is included in the frame 40, in the frame 40 is recognized by the lumen recognition model 92 obtained by the machine learning using the training data 128A generated in the manner shown in FIGS. 7 to 9. As a result, the endoscope apparatus 10 according to the present embodiment enables the doctor 12 or the like to ascertain the position of the lumen 42, which is included in the frame 40, in the frame 40 with higher accuracy, as compared to a case where the position of the lumen 42, which is included in the frame 40, in the frame 40 is recognized by the lumen recognition model 92 obtained by the machine learning using the training data 128A generated in the manner illustrated in FIGS. 7 to 9.

In addition, in the present embodiment, in a case where the certainty information 154 is information in which the value exceeding the third threshold value TH3 is given as the certainty 158 to the first divided region 160A1, the third divided region 160A3, the fifth divided region 160A5, and the seventh divided region 160A7 disposed at regular intervals around the entire circumference of the map 156 among the first to eighth divided regions 160A1 to 160A8 (see FIG. 18), the second visible information 164 is displayed in the first display region 35A. This enables the user or the like to ascertain that the lumen 42 is present in the central region of the frame 40.

In addition, in the present embodiment, in a case where the certainty information 154 is information in which the value exceeding the fourth threshold value TH4 is given as the certainty 158 to the first to eighth divided regions 160A1 to 160A8 (see FIG. 19), the second visible information 164 is displayed in the first display region 35A. This enables the user or the like to ascertain that the lumen 42 is present in the central region of the frame 40.

In addition, in the present embodiment, the first to eighth divided regions 160A1 to 160A8 are regions obtained by radially dividing the frame 40, and the certainty 158 is given to each of the first to eighth divided regions 160A1 to 160A8 to generate the certainty information 154. Then, the first visible information 162 or the second visible information 164 is generated on the basis of the certainty information 154 and is displayed on the screen 35. This enables the user or the like to ascertain the position of the lumen 42 included in the frame 40 in units of the divided regions 160A.

Further, in the present embodiment, in a case where the lumen 42 is included in a region other than the central region of the frame 40, the first visible information 162 and the text 44A are displayed on the screen 35. This enables the user or the like to visually recognize that the lumen 42 is present in the region other than the central region of the frame 40.

Furthermore, in the present embodiment, in a case where the lumen 42 is included in the central region of the frame 40, the first visible information 164 and the text 44B are displayed on the screen 35. This enables the user or the like to visually ascertain that the lumen 42 is present in the central region of the frame 40.

In addition, in the present embodiment, the training data 128A used for the machine learning performed on the model 142 includes the example image 122A2 and the correct answer data 126 associated with the example image 122A2. The image divided into the first to eighth divided regions 150A1 to 150A8 corresponding to the first to eighth divided regions 160A1 to 160A8 is used as the example image 122A2. The correct answer data 126 in a case where the lumen 138 is included in the region other than the central region of the example image 122A2 is an annotation capable of specifying the position of the divided region 150A including the lumen 138. That is, in a case where the lumen 138 is included in the region other than the central region of the example image 122A2, the correct answer data 126 is associated with the divided region 150A including the lumen 138. On the other hand, the correct answer data 126 in a case where the lumen 138 is included in the central region of the example image 122A2 is an annotation capable of specifying the positions of all of the first to eighth divided regions 150A1 to 150A8. That is, in a case where the lumen 138 is included in the region other than the central region of the example image 122A2, the correct answer data 126 is associated with each of the first to eighth divided regions 150A1 to 150A8. In the present embodiment, since the position of the lumen 42, which is included in the frame 40, in the frame 40 is recognized by the lumen recognition model 92 generated by performing the machine learning using the training data 128A obtained in this way on the model 142, the user or the like can ascertain the position of the lumen 42, which is included in the frame 40, in the frame 40 with high accuracy.

Further, in the above-described embodiment, a form is given as an example in which the first visible information 162 is not displayed on the screen 35 and the second visible information 164 is displayed on the screen 35 in a case where the certainty information 154 is the type of information illustrated in FIGS. 17 and 18. However, this is only an example. For instance, in a case where the certainty information 154 is the type of information illustrated in FIGS. 17 and 18, both the first visible information 162 and the second visible information 164 may not be output (for example, both the first visible information 162 and the second visible information 164 may not be displayed on the screen 35). In this way, the user or the like can recognize that neither the first visible information 162 nor the second visible information 164 has been output. As a result, the user or the like can understand that the lumen 42 is present in the central region of the frame 40.

In addition, in the above-described embodiment, the mark that surrounds the outer periphery of the image region (the fan-shaped image region in the example illustrated in FIG. 16) corresponding to the divided region 160A among all of the image regions of the frame 40 is given as an example of the first visible information 162. However, this is only an example, and any information may be used as long as it can visually specify which divided region 160A the lumen 42 is included in.

In addition, in the above-described embodiment, the mark that surrounds the outer periphery of the central region (for example, the image region corresponding to the central region 130A1 illustrated in FIG. 9) among all of the image regions of the frame 40 is given as an example of the second visible information 164. However, this is only an example, and any information may be used as long as it can visually specify that the lumen 42 is included in the central region among all of the image regions of the frame 40.

In addition, in the above-described embodiment, the form in which the example image 122A2 is divided into eight divided regions 150A is given as an example. However, this is only an example, and the example image 122A2 may be divided into nine or more or seven or less divided regions 150A. In this case, the frame 40 may be divided into the same number of divided regions 160A as the divided regions 150A.

In addition, in the above-described embodiment, the information in which the value exceeding the second threshold value TH2 is given as the certainty 158 to the first divided region 160A1 and the fourth divided region 160A4 having the positional relationship exceeding 90 degrees in the circumferential direction CD3 of the map 156 among the first to eighth divided regions 160A1 to 160A8 is given as an example of one of the certainty information items 154. However, the present disclosure is not limited to this. For example, information in which the value exceeding the second threshold value TH2 is given as the certainty 158 to two or more divided regions 160A having the positional relationship of 120 degrees or more in the circumferential direction CD3 of the map 156 among the first to eighth divided regions 160A1 to 160A8 may be used as one of the certainty information items 154. Even in this case, as in the example illustrated in FIG. 16, the first visible information 162 and/or the text 44A is displayed on the screen 35, which makes it possible to obtain the same effect as that in the above-described embodiment.

In addition, in the above-described embodiment, the form in which the second visible information 164 and the text 44B are displayed on the screen 35 in a case where the certainty information 154 is the information of the type illustrated in FIGS. 17 to 19 is given as an example. However, this is only an example. For example, in a case where the certainty information 154 is information in which the value exceeding the second threshold value TH2 is given as the certainty 158 to two or more divided regions 160A, which are disposed at equal intervals around the center of the map 156 and have a positional relationship exceeding 90 degrees in the circumferential direction CD3 of the map 156, among all of the divided regions 160A, the second visible information 164 and/or the text 44B may be displayed on the screen 35.

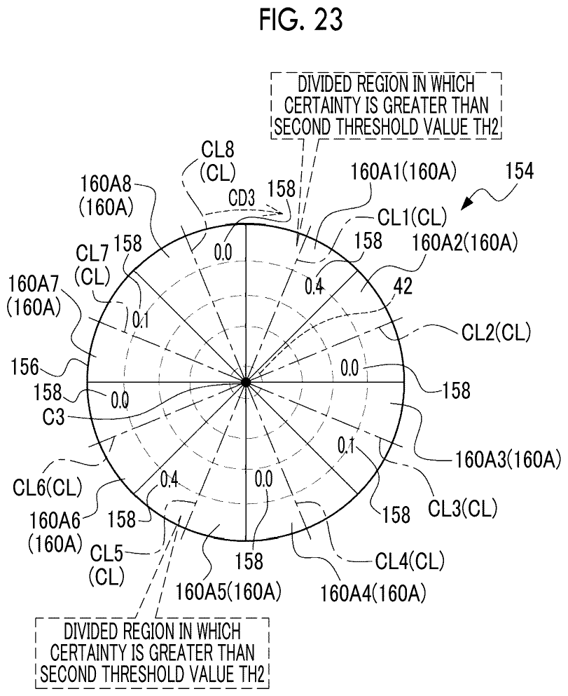

An example of the two or more divided regions 160A which are disposed at equal intervals around the center of the map 156 and have the positional relationship exceeding 90 degrees in the circumferential direction CD3 of the map 156 among all of the divided regions 160A are the first divided region 160A1 and the fifth divided region 150A5 to which a value (for example, 0.4) exceeding the second threshold value TH2 is given as the certainty 158 as illustrated in FIG. 23. In the example illustrated in FIG. 23, since the angle formed between the center line CL1 and the center line CL5 is 180 degrees, it can be said that the positional relationship between the first divided region 160A1 and the fifth divided region 160A5 is the positional relationship in which the divided regions are separated at equal intervals along the circumferential direction CD3 of the map 156. As described above, even in a case where the certainty information 154 is the information of the type illustrated in FIG. 23, it is possible to obtain the same effect as that in the above-described embodiment.

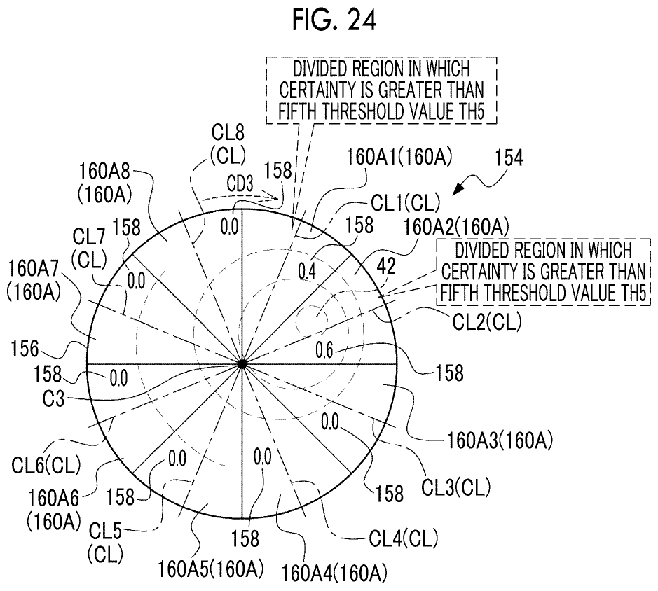

In addition, in the above-described embodiment, the form in which the first visible information 162 and the text 44A are displayed on the screen 35 in a case where the certainty 158 exceeding the first threshold value TH1 is given to a single divided region 160A is given as an example. However, this is only an example. For example, in a case where the certainty information 154 is information in which a value exceeding the fifth threshold value TH5 (for example, 0.3) is given to two or more divided regions 160A having a positional relationship of 90 degrees or less in the circumferential direction CD3 of the map 156 among all of the divided regions 160A, the first visible information 162 and/or the text 44A may be displayed on the screen 35. For example, as illustrated in FIG. 24, since the angle formed between the center line CL1 and the center line CL2 is 45 degrees, it can be said that the positional relationship between the first divided region 160A1 and the second divided region 160A2 is the positional relationship of 90 degrees or less in the circumferential direction CD3 of the map 156. As described above, in a case where the value exceeding the fifth threshold value TH5 is given to the first divided region 160A1 and the second divided region 160A2, for example, a mark that surrounds the outer periphery of a fan-shaped image region (that is, a fan-shaped image region having a central angle of 90 degrees) obtained by combining the first divided region 160A1 and the second divided region 160A2 among all of the image regions of the frame 40 may be displayed as the second visible information 164 to be superimposed on the frame 40. In this case, it is also possible to obtain the same effect as that in the above-described embodiment. In addition, the fifth threshold value TH5 is an example of a “threshold value” according to the present disclosure, and the value exceeding the fifth threshold value TH5 is an example of a “value exceeding the threshold value” according to the present disclosure.

In the above-described embodiment, the form in which the controller 82B outputs the visually specifiable information, such as the text 44A, the text 44B, the first visible information 162, and the second visible information 164, is given as an example. However, the present disclosure is not limited to this. For example, audible information (for example, voice) capable of specifying the position of the lumen 42 in the frame 40 may be output to a speaker (not illustrated), or information capable of specifying the position of the lumen 42 in the frame 40 may be stored in a storage medium (for example, the storage 76, the storage 86, or a storage provided in an external device such as the server).

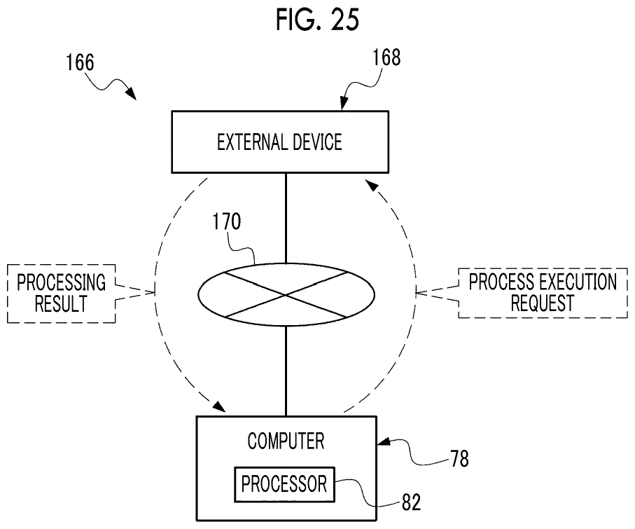

In the above-described embodiment, the form in which the medical support process is performed by the computer 78 has been described as an example. However, the present disclosure is not limited to this. At least some processes included in the medical support process may be performed by a device provided outside the computer 78. Hereinafter, an example of this case will be described with reference to FIG. 25.

FIG. 25 is a conceptual diagram illustrating an example of a configuration of an endoscope apparatus 166. The endoscope apparatus 166 is an example of an “endoscope apparatus” according to the present disclosure. The endoscope apparatus 166 is different from the endoscope apparatus 10 according to the above-described embodiment in that it has an external device 168.

For example, the external device 168 is a server and is connected to the computer 78 via a network 170 (for example, a WAN and/or a LAN) such that it can communicate with the computer 78. Here, the server is given as an example. However, instead of the server, at least one personal computer or the like may be used as the external device 168.

An example of the external device 168 is at least one server that directly or indirectly transmits and receives data to and from the computer 78 via the network 170. The external device 168 receives a process execution instruction given from the processor 82 of the computer 78 via the network 170. Then, the external device 168 executes a process corresponding to the received process execution instruction and transmits a processing result to the computer 78 via the network 170. In the computer 78, the processor 82 receives the processing result transmitted from the external device 168 via the network 170 and executes a process using the received processing result.

An example of the process execution instruction is an instruction for the external device 168 to execute at least a portion of the medical support process. A first example of the at least a portion (that is, a process to be executed by the external device 168) of the medical support process is the lumen recognition process 152. In this case, the external device 168 executes the lumen recognition process 152 in response to the process execution instruction given from the processor 82 via the network 170 and transmits information including the certainty information 154 as a first processing result to the computer 78 via the network 170. In the computer 78, the processor 82 receives the first processing result and executes the same process as that in the above-described embodiment using the received first processing result.

A second example of the at least a portion of the medical support process (that is, the process to be executed by the external device 168) is the process by the controller 82B. In this case, the external device 168 executes the process by the controller 82B in response to the process execution instruction given from the processor 82 via the network 170 and transmits a second processing result (for example, the text 44A, the text 44B, the first visible information 162, and/or the second visible information 164) to the computer 78 via the network 170. In the computer 78, the processor 82 receives the second processing result and executes the same process (for example, the display using the display device 18) as that in the above-described embodiment using the received second processing result.

In addition, the external device 168 may be implemented by cloud computing. The cloud computing is only an example, and the external device 168 may be implemented by network computing, such as fog computing, edge computing, or grid computing.

In the above-described embodiment, the form in which the medical support program 90 is stored in the storage 86 has been described as an example. However, the present disclosure is not limited to this. For example, the medical support program 90 may be stored in a portable non-transitory computer-readable storage medium, such as an SSD or a USB memory. The medical support program 90 stored in the non-transitory storage medium is installed in the computer 78 of the endoscope apparatus 10. The processor 82 executes the medical support process according to the medical support program 90.

In addition, the medical support program 90 may be stored in a storage device of another computer, server, or the like connected to the endoscope apparatus 10 via a network. The medical support program 90 may be downloaded and installed in the computer 78 in response to a request from the endoscope apparatus 10.

In addition, it is not necessary to store all of the medical support program 90 in a storage device of another computer, a server, or the like connected to the endoscope apparatus 10 or to store all of the medical support program 90 in the storage 86. A portion of the medical support program 90 may be stored.

The following various processors can be used as hardware resources for executing the medical support process. An example of the processor is a CPU which is a general-purpose processor that executes software, that is, a program, to function as the hardware resource for executing the medical support process. In addition, an example of the processor is a dedicated electric circuit which is a processor having a dedicated circuit configuration designed to perform a specific process, such as an FPGA, a PLD, or an ASIC. Any processor has a memory provided therein or connected thereto. Any processor uses the memory to perform the medical support process.

The hardware resource for executing the medical support process may be configured by one of the various processors or by a combination of two or more processors of the same type or different types (for example, a combination of a plurality of FPGAs or a combination of a CPU and an FPGA). Further, the hardware resource for executing the medical support process may be one processor.

A first example of the configuration in which the hardware resource is configured by one processor is an aspect in which one processor is configured by a combination of one or more CPUs and software and functions as the hardware resource for performing the medical support process. A second example of the configuration is an aspect in which a processor that implements the functions of the entire system including a plurality of hardware resources for executing the medical support process using one IC chip is used. A representative example of this aspect is an SoC. As described above, the medical support process is achieved using one or more of the various processors as the hardware resource.

In addition, specifically, an electronic circuit obtained by combining circuit elements, such as semiconductor elements, can be used as the hardware structure of the various processors. Further, the above-described medical support process is only an example. Therefore, it goes without saying that unnecessary steps may be deleted, new steps may be added, or the processing order may be changed, without departing from the gist.

The contents described and illustrated above are detailed descriptions of portions related to the present disclosure and are only examples of the present disclosure. For example, the description related to the above configurations, functions, operations, and effects is a description related to examples of configurations, functions, operations, and effects of the portions according to the present disclosure. Therefore, it goes without saying that unnecessary portions may be deleted or new elements may be added or replaced in the content described and illustrated above, without departing from the gist of the present disclosure. Further, in order to avoid complications and to easily understand the portions according to the present disclosure, in the content described and illustrated above, common technical knowledge and the like that do not need to be described to implement the present disclosure are not described.

All of the documents, the patent applications, and the technical standards described in the present specification are incorporated by reference herein to the same extent as each individual document, each patent application, and each technical standard is specifically and individually stated to be incorporated by reference.

Further, the following supplementary notes will be disclosed with respect to the above-described embodiment.

### Supplementary Note 1

A training data generation method for associating correct answer data with an example image to generate training data, the example image being an image indicating a sample of a medical image (for example, the frame 40), the example image being an image divided into a plurality of divided regions (for example, the first to eighth divided regions 150A1 to 150A8) along a circumferential direction, the training data generation method comprising:
in a case where a sample (for example, the lumen 138) of a lumen is included in a region other than a central region of the example image (for example, the example image 122A2), associating the correct answer data (for example, the correct answer data 126) with a divided region including the sample of the lumen among the plurality of divided regions; and
in a case where the sample of the lumen is included in the central region of the example image, associating the correct answer data with each of the plurality of divided regions.

### Supplementary Note 2

The training data generation method according to Supplementary Note 1, in which each of the plurality of divided regions is a region obtained by radially dividing the example image.

### Supplementary Note 3

The training data generation method according to Supplementary Note 2, in which the plurality of divided regions are eight divided regions that are present in a radial shape.

### Supplementary Note 4

A trained model generation method comprising:
in a case where a sample (for example, the lumen 138) of a lumen is included in a region other than a central region of an example image (for example, the example image 122A2) which indicates a sample of a medical image (for example, the frame 40) and is divided into a plurality of divided regions (for example, the first to eighth divided regions 150A1 to 150A8) along a circumferential direction, associating correct answer data (for example, the correct answer data 126) with a divided region including the sample of the lumen among the plurality of divided regions;
in a case where the sample of the lumen is included in the central region of the example image, associating the correct answer data with each of the plurality of divided regions; and
executing machine learning using training data (for example, the training data 128B) obtained by associating the correct answer data with the example image on a model (for example, the model 142) to optimize the model, thereby generating a trained model (for example, the lumen recognition model 92).

### Supplementary Note 5

The trained model generation method according to Supplementary Note 4, in which each of the plurality of divided regions is a region obtained by radially dividing the example image.

### Supplementary Note 6

The trained model generation method according to Supplementary Note 5, in which the plurality of divided regions are eight divided regions that are present in a radial shape.

### Supplementary Note 7

A medical support device comprising:
a processor,
in which the processor is configured to:
input a medical image generated by imaging an inside of a luminal organ including a lumen to a trained model to generate certainty information in which a certainty of the lumen being present in each of a plurality of divided regions obtained by dividing the medical image or an image corresponding to the medical image in a circumferential direction is given to the plurality of divided regions;
output first information indicating that the lumen is present in any of the plurality of divided regions on the basis of the certainty information; and
output second information indicating that the lumen is present in a central region of the medical image in a case where the certainty information is information in which a value exceeding a threshold value is given as the certainty to two or more divided regions having a positional relationship exceeding 90 degrees in the circumferential direction among the plurality of divided regions.

### Supplementary Note 8

The medical support device according to Supplementary Note 7, in which the processor is configured to output the second information in a case where the certainty information is information in which the value is given to two or more divided regions that are disposed at equal intervals around a center of the medical image or the image and have a positional relationship exceeding 90 degrees in the circumferential direction among the plurality of divided regions.

### Supplementary Note 9

The medical support device according to Supplementary Note 7, in which the processor is configured to output the second information in a case where the certainty information is information in which the value is given to two or more divided regions having a positional relationship of 120 degrees or more in the circumferential direction among the plurality of divided regions.

### Supplementary Note 10

The medical support device according to Supplementary Note 7, in which the processor is configured to output the second information in a case where the certainty information is information in which the value is given to two or more divided regions disposed at regular intervals around an entire circumference of the medical image or the image among the plurality of divided regions.

### Supplementary Note 11

The medical support device according to Supplementary Note 7, in which the processor is configured to output the second information in a case where the certainty information is information in which the value is given to all of the plurality of divided regions.

### Supplementary Note 12

The medical support device according to any one of Supplementary Note 7 to Supplementary Note 11, in which the processor is configured to output the first information in a case where the certainty information is information in which the value is given to a single divided region among the plurality of divided regions and in a case where the certainty information is information in which the value is given to two or more divided regions having a positional relationship of 90 degrees or less in the circumferential direction among the plurality of divided regions.

### Supplementary Note 13

The medical support device according to any one of Supplementary Note 7 to Supplementary Note 12, in which each of the plurality of divided regions is a region obtained by radially dividing the medical image.

### Supplementary Note 14

The medical support device according to Supplementary Note 13, in which the plurality of divided regions are eight divided regions that are present in a radial shape.

### Supplementary Note 15

The medical support device according to any one of Supplementary Note 7 to Supplementary Note 14, in which the trained model is obtained by machine learning using training data including an example image, which indicates a sample of the medical image and is divided into a plurality of regions corresponding to the plurality of divided regions, and correct answer data associated with the example image, the correct answer data in a case where a sample of the lumen is included in a region other than a central region of the example image may be an annotation capable of specifying a position of the region including the sample of the lumen among the plurality of regions, and the correct answer data in a case where the sample of the lumen is included in the central region of the example image may be an annotation capable of specifying positions of all of the plurality of regions.

### Supplementary Note 16

The medical support device according to any one of Supplementary Note 7 to Supplementary Note 15, in which the processor is configured to: display the first information on a screen to output the first information; and display the second information on the screen to output the second information.

### Supplementary Note 17

The medical support device according to Supplementary Note 16, in which the processor is configured to: display the medical image on the screen; display the first information in the medical image displayed on the screen; and display the second information in the medical image displayed on the screen.

### Supplementary Note 18

The medical support device according to any one of Supplementary Note 7 to Supplementary Note 17, in which the first information and the second information are visible information capable of visually specifying a position of the lumen included in the medical image.

### Supplementary Note 19

The medical support device according to any one of Supplementary Note 7 to Supplementary Note 18, in which the medical image is an endoscope image generated by imaging the inside of the luminal organ including the lumen with an endoscope.

### Supplementary Note 20

An endoscope apparatus comprising:
the medical support device according to any one of Supplementary Note 7 to Supplementary Note 19; and an endoscope, in which the medical image is generated by imaging the inside of the luminal organ including the lumen with the endoscope.

### Supplementary Note 21

A medical support method comprising:
inputting a medical image generated by imaging an inside of a luminal organ including a lumen to a trained model to generate certainty information in which a certainty of the lumen being present in each of a plurality of divided regions obtained by dividing the medical image or an image corresponding to the medical image in a circumferential direction is given to the plurality of divided regions;
outputting first information indicating that the lumen is present in any of the plurality of divided regions on the basis of the certainty information; and
outputting second information indicating that the lumen is present in a central region of the medical image in a case where the certainty information is information in which a value exceeding a threshold value is given as the certainty to two or more divided regions having a positional relationship exceeding 90 degrees in the circumferential direction among the plurality of divided regions.

### Supplementary Note 22

A program causing a computer to execute a process comprising:
inputting a medical image generated by imaging an inside of a luminal organ including a lumen to a trained model to generate certainty information in which a certainty of the lumen being present in each of a plurality of divided regions obtained by dividing the medical image or an image corresponding to the medical image in a circumferential direction is given to the plurality of divided regions;
outputting first information indicating that the lumen is present in any of the plurality of divided regions on the basis of the certainty information; and
outputting second information indicating that the lumen is present in a central region of the medical image in a case where the certainty information is information in which a value exceeding a threshold value is given as the certainty to two or more divided regions having a positional relationship exceeding 90 degrees in the circumferential direction among the plurality of divided regions.

What is claimed is:

1. A medical support device comprising a processor,
wherein the processor is configured to:
input a medical image generated by imaging an inside of a luminal organ including a lumen to a trained model to generate certainty information in which a certainty of the lumen being present in each of a plurality of divided regions obtained by dividing the medical image or an image corresponding to the medical image in a circumferential direction is given to the plurality of divided regions; and
perform an output process capable of distinguishing between a case where the certainty information is not information in which a value exceeding a threshold value is given as the certainty to two or more divided regions having a positional relationship exceeding 90 degrees in the circumferential direction among the plurality of divided regions, and a case where the certainty information is information in which a value exceeding the threshold value is given as the certainty to two or more divided regions having a positional relationship exceeding 90 degrees in the circumferential direction among the plurality of divided regions.

2. The medical support device according to claim 1, wherein:
the output process includes outputting first information indicating that the lumen is present in any of the plurality of divided regions based on the certainty information in a case where the certainty information is not information in which a value exceeding the threshold value is given as the certainty to two or more divided regions having a positional relationship exceeding 90 degrees in the circumferential direction among the plurality of divided regions, and not outputting the first information in a case where the certainty information is information in which a value exceeding the threshold value is given as the certainty to two or more divided regions having a positional relationship exceeding 90 degrees in the circumferential direction among the plurality of divided regions.

3. The medical support device according to claim 1, wherein:
the output process includes outputting second information indicating that the lumen is present in a central region of the medical image, or not outputting the second information, in a case where the certainty information is information in which a value exceeding the threshold value is given as the certainty to two or more divided regions having a positional relationship exceeding 90 degrees in the circumferential direction among the plurality of divided regions.

4. The medical support device according to claim 3, wherein:
the output process includes not outputting the second information in a case where the certainty information is not information in which a value exceeding the threshold value is given as the certainty to two or more divided regions having a positional relationship exceeding 90 degrees in the circumferential direction among the plurality of divided regions.

5. The medical support device according to claim 3, wherein:
the output process includes outputting the second information in a case where the certainty information is information in which the value is given to two or more divided regions that are disposed at equal intervals around a center of the medical image or the image and have a positional relationship exceeding 90 degrees in the circumferential direction among the plurality of divided regions.

6. The medical support device according to claim 3, wherein:
the output process includes outputting the second information in a case where the certainty information is information in which the value is given to two or more divided regions having a positional relationship of 120 degrees or more in the circumferential direction among the plurality of divided regions.

7. The medical support device according to claim 3, wherein:
the output process includes outputting the second information in a case where the certainty information is information in which the value is given to two or more divided regions disposed at regular intervals around an entire circumference of the medical image or the image among the plurality of divided regions.

8. The medical support device according to claim 3, wherein:
the output process includes outputting the second information in a case where the certainty information is information in which the value is given to all of the plurality of divided regions.

9. The medical support device according to claim 2, wherein:
the output process includes outputting the first information in a case where the certainty information is information in which the value is given to a single divided region among the plurality of divided regions and in a case where the certainty information is information in which the value is given to two or more divided regions having a positional relationship of 90 degrees or less in the circumferential direction among the plurality of divided regions.

10. The medical support device according to claim 1, wherein:
each of the plurality of divided regions is a region obtained by radially dividing the medical image.

11. The medical support device according to claim 10, wherein:
the plurality of divided regions are eight divided regions that are present in a radial shape.

12. The medical support device according to claim 1, wherein:
the trained model is obtained by machine learning using training data including an example image, which indicates a sample of the medical image and is divided into a plurality of regions corresponding to the plurality of divided regions, and correct answer data associated with the example image,
the correct answer data in a case where a sample of the lumen is included in a region other than a central region of the example image is an annotation capable of specifying a position of the region including the sample of the lumen among the plurality of regions, and
the correct answer data in a case where the sample of the lumen is included in the central region of the example image is an annotation capable of specifying positions of all of the plurality of regions.

13. The medical support device according to claim 1, wherein:
the output process includes:
displaying first information indicating that the lumen is present in any of the plurality of divided regions based on the certainty information on a screen to output the first information in a case where the certainty information is not information in which a value exceeding a threshold value is given as the certainty to two or more divided regions having a positional relationship exceeding 90 degrees in the circumferential direction among the plurality of divided regions; and
displaying second information indicating that the lumen is present in a central region of the medical image on the screen to output the second information in a case where the certainty information is information in which a value exceeding a threshold value is given as the certainty to two or more divided regions having a positional relationship exceeding 90 degrees in the circumferential direction among the plurality of divided regions.

14. The medical support device according to claim 13, wherein:
the processor is configured to:
display the medical image on the screen;
display the first information in the medical image displayed on the screen; and
display the second information in the medical image displayed on the screen.

15. The medical support device according to claim 1, wherein:
the output process includes outputting first information indicating that the lumen is present in any of the plurality of divided regions based on the certainty information in a case where the certainty information is not information in which a value exceeding the threshold value is given as the certainty to two or more divided regions having a positional relationship exceeding 90 degrees in the circumferential direction among the plurality of divided regions, and outputting second information indicating that the lumen is present in a central region of the medical image in a case where the certainty information is information in which a value exceeding the threshold value is given as the certainty to two or more divided regions having a positional relationship exceeding 90 degrees in the circumferential direction among the plurality of divided regions; and
the first information and the second information are visible information capable of visually specifying a position of the lumen included in the medical image.

16. The medical support device according to claim 1, wherein:
the medical image is an endoscope image generated by imaging the inside of the luminal organ including the lumen with an endoscope.

17. An endoscope apparatus comprising:
the medical support device according to claim 1; and
an endoscope,
wherein the medical image is generated by imaging the inside of the luminal organ including the lumen with the endoscope.

18. A medical support method comprising:
inputting a medical image generated by imaging an inside of a luminal organ including a lumen to a trained model to generate certainty information in which a certainty of the lumen being present in each of a plurality of divided regions obtained by dividing the medical image or an image corresponding to the medical image in a circumferential direction is given to the plurality of divided regions; and
performing an output process capable of distinguishing between a case where the certainty information is not information in which a value exceeding a threshold value is given as the certainty to two or more divided regions having a positional relationship exceeding 90 degrees in the circumferential direction among the plurality of divided regions, and a case where the certainty information is information in which a value exceeding the threshold value is given as the certainty to two or more divided regions having a positional relationship exceeding 90 degrees in the circumferential direction among the plurality of divided regions.

19. A non-transitory computer-readable storage medium storing a program executable by a computer to execute a process comprising:
inputting a medical image generated by imaging an inside of a luminal organ including a lumen to a trained model to generate certainty information in which a certainty of the lumen being present in each of a plurality of divided regions obtained by dividing the medical image or an image corresponding to the medical image in a circumferential direction is given to the plurality of divided regions; and
performing an output process capable of distinguishing between a case where the certainty information is not information in which a value exceeding a threshold value is given as the certainty to two or more divided regions having a positional relationship exceeding 90 degrees in the circumferential direction among the plurality of divided regions, and a case where the certainty information is information in which a value exceeding the threshold value is given as the certainty to two or more divided regions having a positional relationship exceeding 90 degrees in the circumferential direction among the plurality of divided regions.
